(19) United States
(12) Patent Application Publication
(10) Pub. No.: US 2016/0015893 A1
(43) Pub. Date: Jan. 21, 2016

(54) DIRECT PRESSURE-MEDIATED INTRA-BONE DELIVERY SYSTEM FOR CELLULAR THERAPEUTICS

(71) Applicant: The United States of America, as represented by the Secretary, Dept. of Health & Human Services, Rockville, MD (US)

(72) Inventors: Robert Franklin Hoyt, Boyds, MD (US); Jeremy Mark Pantin, Augusta, GA (US); Omer Aras, New York, NY (US); Richard William Childs, Rockville, MD (US); Peter Choyke, North Bethesda, MD (US); Randall Ray Clevenger, Mount Airy, MD (US); Timothy John Hunt, Damascus, MD (US); Jan Davidson-Moncada, Chevy Chase, MD (US)

(21) Appl. No.: 14/772,044

(22) PCT Filed: Feb. 28, 2014
(86) PCT No.: PCT/US2014/019401

§ 371 Date: Sep. 1, 2015

Related U.S. Application Data
(60) Provisional application No. 61/771,463, filed on Mar. 1, 2013.

(51) Int. Cl. A61M 5/172; A61B 17/16; A61B 17/34

(52) U.S. Cl. CPC A61M 5/1723; A61B 17/3472; A61B 17/3494; A61B 17/16; A61B 2017/3458; A61B 2017/00469

(57) ABSTRACT

Disclosed are devices, apparatus, and methods for directly infusing one or more materials into a bone of a patient. More particularly, devices, apparatus and methods are provided for direct intra-bone infusion, wherein intra-bone pressure is continuously monitored and adjusted during infusion such that intra-bone pressure does not exceed levels of systemic blood pressure. Such devices, apparatus and methods are particularly suitable for use in performing bone marrow transplants.

### RELATED APPLICATIONS

This application claims priority to U.S. Provisional Application No. 61/771,463, filed on Mar. 1, 2013, and International Patent Application Serial No. PCT/US2014/019401 filed on Feb. 28, 2014 the contents of which are incorporated herein by referenced in their entirety.

### GOVERNMENT SUPPORT

This invention was funded by the National Institutes of Health. The United States Government has certain rights in this invention.

### FIELD OF INVENTION

The present invention generally relates to techniques and devices for infusing materials into bone marrow, particularly wherein intra-bone pressure is continuously monitored and adjusted during infusion such that intra-bone pressure does not exceed a desired pressure.

### BACKGROUND OF THE INVENTION

Allogeneic hematopoietic cell transplantation (HCT) is an effective treatment for a variety of hematological diseases. Intravenous (IV) injection is the routine method for HCT; however, intrabone (IB) HCT has been reported to have some advantages regarding engraftment and lower incidence of graft-versus-host disease (GVHD). To date, IB HCT transplantation has been investigated pre-clinically in small mammals and in a limited number of human trials. Impediments to the progress of IB transplantation include uncertainty regarding the optimized injection conditions that result in the best long-term outcomes for HCT. It is not known in humans whether hematopoietic progenitor cells (HPCs) injected IB are immediately retained in the marrow or rapidly enter into venous circulation and eventually return back to the marrow. An optimized method for direct IB infusion of HPCs in humans, which maximizes cellular retention in the bone marrow, has not yet been developed. The ability to track HPCs in vivo with radionuclide cell-labeling would provide guidance for injection optimization.

Therefore, a need exists for improved techniques and devices that allow for direct infusion of materials into a bone, particularly in performing a bone marrow transplant, which can reduce and even prevent potential leakage of the materials out of the bone, compartment syndrome and GVHD, and which can increase the levels of engraftment.

### SUMMARY OF THE INVENTION

Transplantation of agents, particularly allogeneic hematopoietic progenitor cells (HPC), is an effective treatment for a variety of hematological diseases. It is an object of the present invention to optimize intra-bone transplantation so as to retain one or more infused agents, such as HPCs, within the target bone marrow into which the agents are infused (e.g. the pelvic bone). It is a further object of the present invention to further provide for methods for assessing the infusion, such as by radionuclide labeling to assess the trafficking of HPCs, using imaging techniques during various stages of the intra-bone transplantation, including early stages after IB transplantation and subsequent thereto.

The present invention features methods, apparatus and devices for direct intra-bone infusion of materials into a patient, and particularly to such methods, apparatus and devices in which stem cells are directly infused into a patient's bone during a bone marrow transplant.

In an embodiment of the present invention, there is featured an intra-bone infusion system for performing a bone marrow transplant comprising an infusion source, an intra-bone device having a pressure sensor at a proximal end, and a mechanism in communication with the pressure sensor and the infusion source. The intra-bone device has a proximal end for insertion into bone marrow, a distal end in connection with the infusion source, and a pressure sensor at the distal end for measuring intra-bone pressure. The mechanism is configured and arranged to continuously monitor intra-bone pressure measurements by the pressure sensor, and to automatically adjust infusion of agents from the infusion source and into the intra-bone device so as to maintain intra-bone pressure at levels not exceeding systemic blood pressure.

Aspects in accordance with this embodiment can include the following features. The intra-bone device can comprise an access cannula and an infusion cannula. The access cannula can have a hub, an elongate portion extending from the hub, and a lumen extending through a length of the elongate portion. The infusion cannula can have a handle and an elongate portion insertable within the lumen of the access cannula. A distal end of the elongate portion of the access cannula can be threaded. A distal end of the infusion cannula can have one or more openings through which agents may be infused, and the elongate portion of the infusion cannula can be hollow. When the infusion cannula is inserted within the lumen of the access cannula, a distal end of the infusion cannula can extend beyond a distal end of the access cannula. The pressure sensor can be disposed along the outer surface of the infusion cannula. The intra-bone device can further comprise a penetrator. The penetrator can have a handle and an elongate portion extending from the handle. The penetrator can be insertable within the lumen of the access cannula. The pressure sensor can be a fiberoptic probe. One or more portions of the intra-bone device can be imagable by MRI, CT, fluoroscopy guidance imaging systems, optical guidance imaging systems, ultrasound guidance imaging systems, and the like. The device can be designed to present minimal artifacts on PET scans. The system can further comprise a robotic guidance system, a CT guidance system, an MRI guidance system, a fluoroscopy guidance imaging system, an optical guidance imaging system, an ultrasound guidance imaging system, and the like.

In another embodiment of the present invention, there is featured a method for performing a bone marrow transplant comprising inserting an intra-bone device into a target bone, infusing one or more agents into the target bone while continuously measuring intra-bone pressure, and adjusting a rate of infusion of the one or more agents to maintain intra-bone pressure at levels not exceeding 25-30 mmHg. The intra-bone device can be provided with any combination of features described herein.

Aspects in accordance with this embodiment can include the following features. The one or more agents can be selected from bone marrow, hematopoetic stem cells, mesenchymal stem cells and gene therapy vectors. The method can be carried out under MRI imaging, CT imaging, fluoroscopy guidance imaging, optical guidance imaging, ultrasound guidance imaging, and the like. One or more of the steps of inserting the intra-bone device, infusing one or more agents, and adjusting a rate of infusion can be carried out robotically.

In one aspect, the present invention features an intra-bone infusion system for performing a bone marrow transplant comprising an infusion source; an intra-bone device having a proximal end for insertion into bone marrow, a distal end in connection with the infusion source, and a pressure sensor at the distal end for measuring intra-bone pressure; and a mechanism in communication with the pressure sensor and the infusion source, the mechanism configured and arranged to continuously monitor intra-bone pressure measurements by the pressure sensor, and to automatically adjust infusion of agents from the infusion source and into the intra-bone device so as to maintain intra-bone pressure at levels not exceeding systemic blood pressure.

In another aspect, the present invention features an intra-bone infusion system for performing a bone marrow transplant comprising: an infusion source; an intra-bone device having a proximal end for insertion into bone marrow, a distal end in connection with the infusion source, and a pressure sensor at the distal end for measuring intra-bone pressure; and a mechanism in communication with the pressure sensor and the infusion source, the mechanism configured and arranged to continuously monitor intra-bone pressure measurements by the pressure sensor, and to automatically adjust infusion of agents from the infusion source and into the intra-bone device so as to maintain intra-bone pressure at levels not exceeding 25-30 mmHg.

In one embodiment of the present invention, the intra-bone device comprises an access cannula and an infusion cannula, wherein the access cannula has a hub, an elongate portion extending from the hub, and a lumen extending through a length of the elongate portion, and the infusion cannula has a handle and an elongate portion insertable within the lumen of the access cannula.

In another embodiment of the present invention, a distal end of the elongate portion of the access cannula is threaded.

In one embodiment of the present invention, a distal end of the infusion cannula has one or more openings through which agents may be infused, and wherein the elongate portion of the infusion cannula is hollow.

In another embodiment of the present invention, when the infusion cannula is inserted within the lumen of the access cannula, a distal end of the infusion cannula extends beyond a distal end of the access cannula. In a related embodiment of the present invention, the pressure sensor is disposed along the outer surface of the infusion cannula.

In one embodiment of the present invention, the intra-bone device further comprises a penetrator, the penetrator having a handle and an elongate portion extending from the handle, wherein the penetrator is insertable within the lumen of the access cannula.

In one embodiment of the present invention, the pressure sensor is a fiberoptic probe.

In another embodiment of the present invention, the intra-bone device is imagable by a number of imaging modalities including MRI, CT, fluoroscopy guidance imaging, optical guidance imaging, ultrasound guidance imaging, and the like.

In one embodiment of the present invention, the intra-bone infusion system further comprises a robotic guidance system, a CT guidance system, an MRI guidance system, a CT imaging system, a fluoroscopy guidance imaging system, an optical guidance imaging system, or an ultrasound guidance imaging system.

In another aspect, the present invention features a method for infusing one or more agents into a target bone comprising: inserting an intra-bone device into a target bone; infusing one or more agents into the target bone while continuously measuring intra-bone pressure; and adjusting a rate of infusion of the one or more agents to maintain intra-bone pressure at levels not exceeding systemic blood pressure.

In another aspect, the present invention features a method for performing a bone marrow transplant comprising: inserting an intra-bone device into a target bone; infusing one or more agents into the target bone while continuously measuring intra-bone pressure; and adjusting a rate of infusion of the one or more agents to maintain intra-bone pressure at levels not exceeding systemic blood pressure.

In one embodiment of the present invention, the one or more agents are selected from bone marrow, hematopoetic stem cells, mesenchymal stem cells, therapeutic agents and gene therapy vectors.

In another aspect, the present invention features a method for improving the rate of retention of hematopoetic stem cells in a bone marrow transplant comprising: inserting an intra-bone device into a target bone; infusing one or more agents into the target bone while continuously measuring intra-bone pressure; and adjusting a rate of infusion of the one or more agents to maintain intra-bone pressure at levels not exceeding systemic blood pressure, thereby improving the rate of retention of hematopoetic stem cells.

In one embodiment of the present invention, the target bone is the pelvic bone.

In another embodiment of the present invention, the method of any one of the above aspects is carried out under MRI imaging, CT imaging, fluoroscopy guidance imaging, optical guidance imaging, ultrasound guidance imaging, and the like.

In one embodiment of the present invention, one or more of the steps of inserting the intra-bone device, infusing one or more agents, and adjusting a rate of infusion are carried out robotically.

In another aspect, the present invention features a method for increasing engraftment in a human subject undergoing a bone marrow transplant comprising: inserting an intra-bone device into a target bone, wherein the intra-bone device is provided with a pressure sensor for measuring intra-bone pressure; infusing one or more agents through the intra-bone device while continuously measuring intra-bone pressure; and adjusting a rate of infusion of the one or more agents to maintain intra-bone pressure at levels not exceeding systemic blood pressure.

In one embodiment of the present invention, at least about 85% engraftment is provided. According to some embodiments, engraftment of at least about 90% and even at least about 95% can be provided.

In another embodiment of the present invention, the infusion volume of the one or more agents is between 5-15 ml.

In one embodiment of the present invention, the rate of infusion is less than 0.2 ml/s.

In one embodiment of the present invention, the method further comprises the step of temporary occlusion of the iliac vein.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “a sensor” includes reference to more than one sensor.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “agent” is meant to include bone marrow, hematopoetic stem cells, mesenchymal stem cells, gene therapy vectors, local chemotherapeutic agents, medications, blood products, and fluids.

By “disease” is meant any condition or disorder that damages or interferes with the normal function of a cell, tissue, or organ. An exemplary disease is leukemia.

By “drug” is meant a chemical compound, composition, agent (e.g., a pharmaceutical agent) capable of inducing a pharmacological effect in a subject. A drug when properly administered to a patient as a pharmaceutical agent has a desired therapeutic effect.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition, e.g., GVHD.

By “reduces” is meant a negative alteration of at least 5%, 10%, 25%, 50%, 75%, or 100%.

By “increases” and is meant a positive alteration of at least 5% 10%, 25%, 50%, 75%, or 100%.

As used herein, the term “sample” includes a biologic sample such as any tissue, cell, fluid, or other material derived from an organism.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

As used herein, the terms “treat,” treating,” “treatment,” and the like refer to reducing or ameliorating a disease or condition, e.g., leukemia, and/or symptoms associated therewith. It will be appreciated that, although not precluded, treating a disease or condition does not require that the disease, condition, or symptoms associated therewith be completely eliminated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, apparatus, or methods provided herein can be combined with one or more of any of the other compounds, compositions, apparatus, and methods provided herein.

### DESCRIPTION OF THE DRAWINGS

FIGS. 1a-1c illustrate the intra-bone device in accordance with one embodiment.

FIG. 1a shows an access cannula, FIG. 1b shows an infusion cannula, and FIG. 1c shows the access cannula disposed within a bone and the infusion cannula inserted through the access cannula into the bone.

FIGS. 2a-2c illustrate an embodiment of an access cannula in accordance with one embodiment. FIG. 2a shows the access cannula, FIG. 2b shows a detailed cross-sectional view of a valve within the access cannula, and FIG. 2c shows the direction in which an infusion cannula may be insertable through the valve of the access cannula.

FIG. 3 illustrates an embodiment of a penetrator being inserted within an access cannula, and a handle that can be removably attached to the penetrator.

FIG. 4a illustrates an access cannula and a penetrator, individually, and the penetrator inserted within the access cannula in accordance with an embodiment.

FIG. 4b illustrates an access cannula and an infusion cannula, individually, and the infusion cannula inserted within the access cannula in accordance with an embodiment.

FIG. 5 shows a schematic of an intra-bone infusion system implanted within a bone, wherein one or more agents are infused in the bone marrow while continuously monitoring and adjusting infusion so as to maintain a desired intra-bone pressure.

FIG. 6 is a set of graphs that show physiologic parameters following IB injection of 10 ml at 1.0 ml/s (rapid) and at 0.1 ml/s (slow). With rapid injection, there is a significant peak in intramarrow pressure followed by an increase in PA pressure, decrease in systemic arterial pressure, and tachycardia. With slower injection, these changes are not observed.

FIG. 7 (a and b) are two graphs that show hCD34+ cells were labeled with neutralized ⁸⁹Zr as described. (a)⁸⁹Zr labeling of hCD34+ cells did not affect the survival of the cells. (b)⁸⁹Zr was gradually released from ⁸⁹Zr-hCD34+-cells.

FIG. 8 (a-e) shows fusion PET/CT coronal images at 1 hr following (a) IB injection of 89Zr-Df with radioactivity in the renal pelvises and bladder; (b) IV infusion of 89Zr-hCD34+ with radioactivity confined to the lung; (c) Hand IB injection of 89Zr-hCD34+ into two sites on the right iliac crest demonstrating radioactivity leaked at the first site of injection (arrow) and radioactivity within the pelvic bone and lungs; (d) Hand IB injection of 89Zr-hCD34+ into the right iliac crest demonstrating radioactivity leaked at the sight of injection (arrow) and radioactivity within the pelvic bone and lungs; (e) slow IB infusion (0.2 ml/min) of 89Zr-hCD34+ into the right iliac crest demonstrating radioactivity confined within the pelvic bone. Note that in (d) there is radioactivity highlighted in syringes of 89Zr-oxalate placed to the left of the swine for calibration and decay correction (star).

FIG. 9 (a and b) are graphs that show systemic arterial and intramarrow pressure over time during (a) hand injection of 89Zr-hCD34+IB into the iliac crest through a single site and (b) slow infusion at 0.2 ml/min of 89Zr-hCD34+ into the iliac crest through a single site.

FIG. 10 shows a shortened ONCONTROL needle with distal fenestrations (arrows) added. The needle was driven directly into the iliac crest to the hub. A longer needle was used for CT-guided percutaneous access (Pig #34489).

FIG. 11 shows simultaneous infusion and intrmarrow pressure monitoring with a Millar catheter through a hemostasis type-Y connector connected to an ONCONTROL needle inserted into the iliac crest.

FIG. 12 shows CT angiography of the left hemipelvic terminal branches of the abdominal aorta in Sus scrofa. The right external and internal iliac arteries were reversibly occluded by external vessel occluders (arrows) during contrast injection.

FIG. 13 shows 3D reconstruction following second IB hand-injection of contrast with two access sites on the right iliac crest. Leakage demonstrated from the first injection site with run-off (arrow).

FIG. 14 (a-c) shows fusion PET/CT coronal images following right iliac artery intra-arterial infusion of 89Zr-hCD34+ at (a) 1 h, (b) 2 h, and (c) 3 h post infusion. Radioactivity is noted to advance from within the pelvic bone into the surrounding muscle in the direction of arterial blood flow.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION OF THE INVENTION

This invention is based, at least in part, on the discovery that intra-bone delivery of agents into bone marrow can be carried out under controlled conditions such that a majority of the delivered agents are retained within the bone marrow rather than escaping into the vascular system. Accordingly, the invention provides apparatus and techniques that are useful in the intra-bone delivery of agents. The invention further provides apparatus and techniques for performing a bone marrow transplant by delivering agents directly into the bone marrow, thereby treating a variety of conditions including acute leukemias, brain tumors, breast cancer, Hodgkin's disease, multiple myeloma, neuroblastoma, non-Hodgkin's lymphomas, ovarian cancer, sarcoma and testicular cancer.

The present methods, apparatus and devices can be used in connection with known imaging systems and techniques to image the internal bodily tissues, organs, structures, cavities, and spaces of the subject being treated. For example, the systems and methods described herein can include transmitter or receiver coils to facilitate active-device navigation using an imaging system, such as magnetic-resonance imaging (MRI), CT imaging, fluoroscopy guidance imaging, optical guidance imaging, ultrasound guidance imaging, and the like. This imaging can be conducted along arbitrary or predetermined planes using various imaging methods based on X-ray technologies, X-ray fluoroscopy, MRI, electromagnetic-position navigation, co-registration of X-ray and MRI or of X-ray and CT, video technologies (such as endoscopy, infra-red imaging, saline-flush videoscopy and the like), ultrasound, and other such technologies. In some embodiments, real-time MRI (rtMRI), intracardiac ultrasound, or electromagnetic guidance is employed. Thus, as used herein, the term “imaging system” includes any device, apparatus, system, or method of imaging the internal regions of a subject's body.

Further, the present methods, apparatus and devices can be advantageously adapted for use in procedures that use automated means for performing the infusion, such as robotic means controlled by an operator.

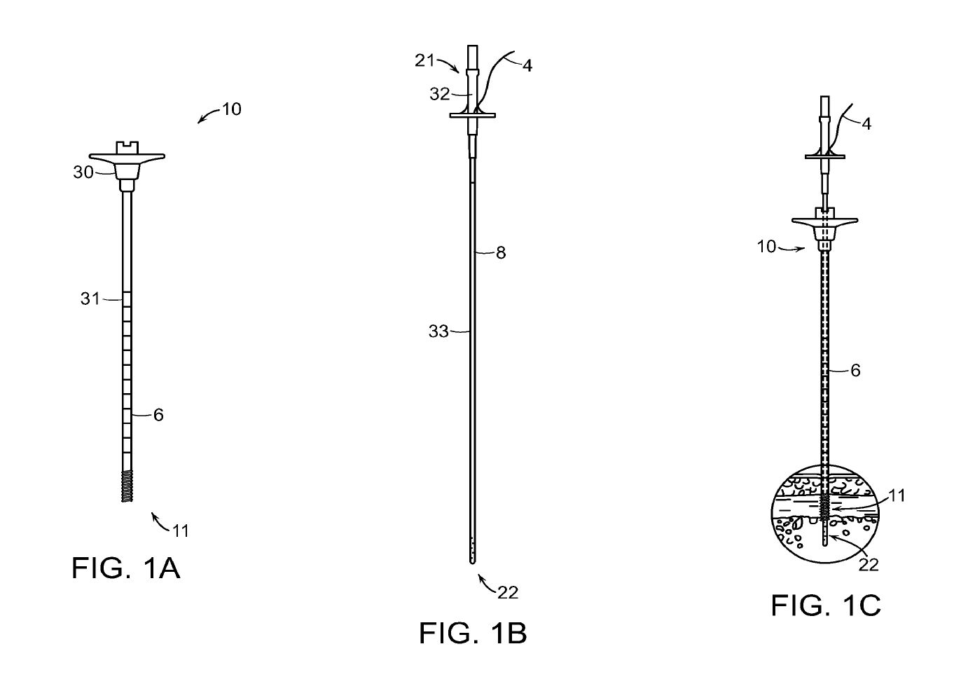

A general embodiment of an intra-bone device 1, is shown in FIGS. 1a-1c, wherein the intra-bone device 1 includes an access cannula 6 and an infusion cannula 8. As shown in FIG. 1c, the infusion cannula 8 is insertable within an inner lumen (not shown) of the access cannula 6. The access cannula 6 includes a proximal end 10 and a distal end 11. As shown in FIG. 1a, the access cannula can be provided with a hub 30 portion and an elongate portion 31. While the elongate portion 31 is preferably tubular in shape with a circular cross-section, any other variety of geometries can also be suitably used. The infusion cannula 8 includes a proximal end 21 and a distal end 22. As shown in FIG. 1b, the infusion cannula 8 can include a handle portion 32 and an elongate portion 33. While the elongate portion 33 of the infusion cannula 8 is preferably tubular in shape with a circular cross-section, any other variety of geometries can also be suitably used which are compatible with the geometry of the access cannula 6 through which the infusion cannula 8 is insertable.

In accordance with this embodiment, the access cannula 6 can, for example, be first disposed within the bone as desired. Thereafter, the infusion cannula 8 can be inserted through the access cannula 6 and position for infusion of one or more agents into the bone marrow. For example, as shown in FIG. 1c, a distal end 22 of the infusion cannula 8 can protrude beyond the distal end of the access cannula 6. It is, of course, possible to insert the infusion cannula 8 through the access cannula 6 without the distal end 22 of the infusion cannula 8 protruding beyond the distal end of the access cannula 6, as long as the one or more agents can be infused through the infusion cannula 8 and into the bone marrow and as long as the intra-bone device 1 is suitably adapted with a pressure sensor 4 for monitoring intra-bone pressure.

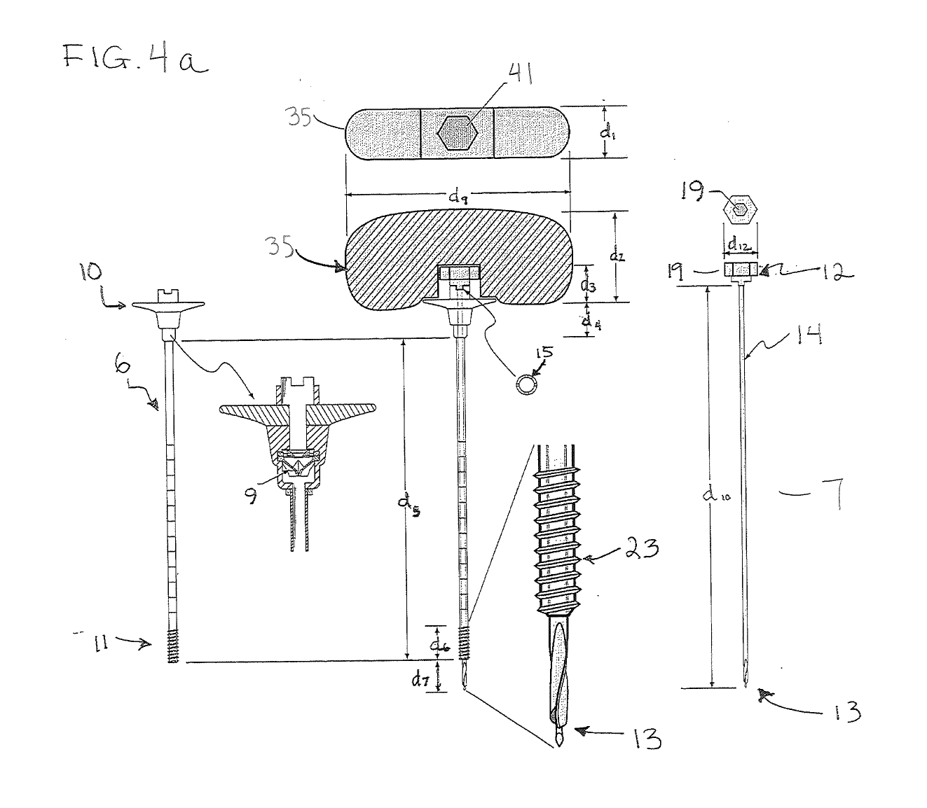

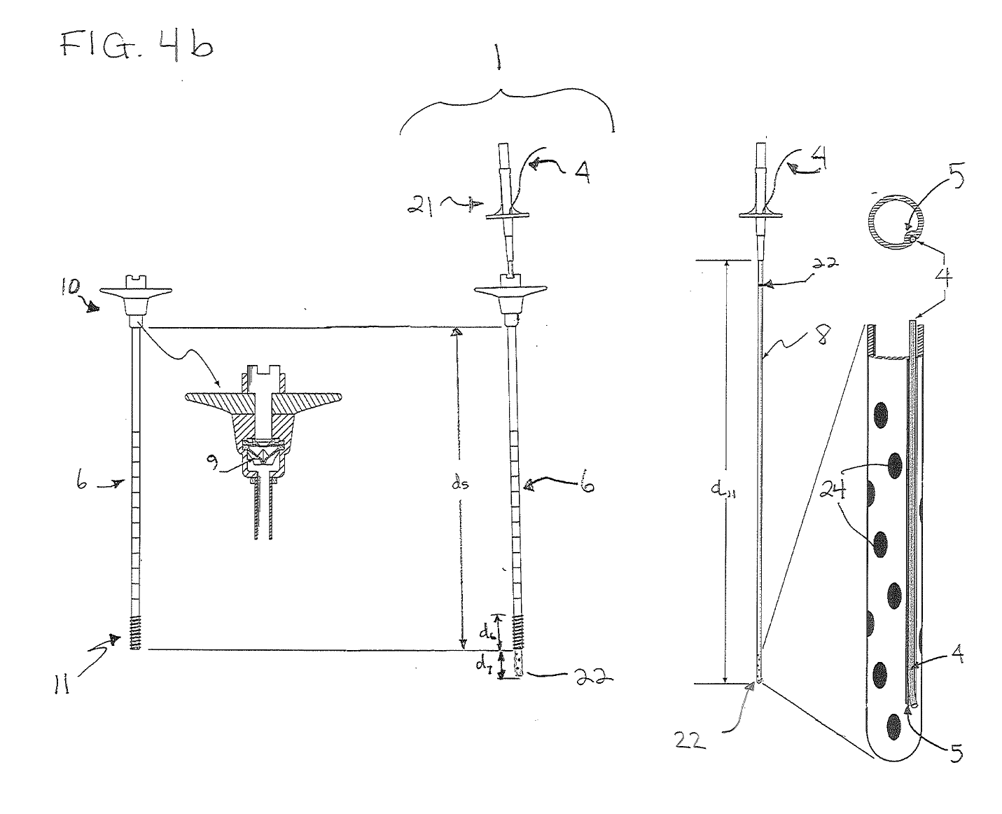

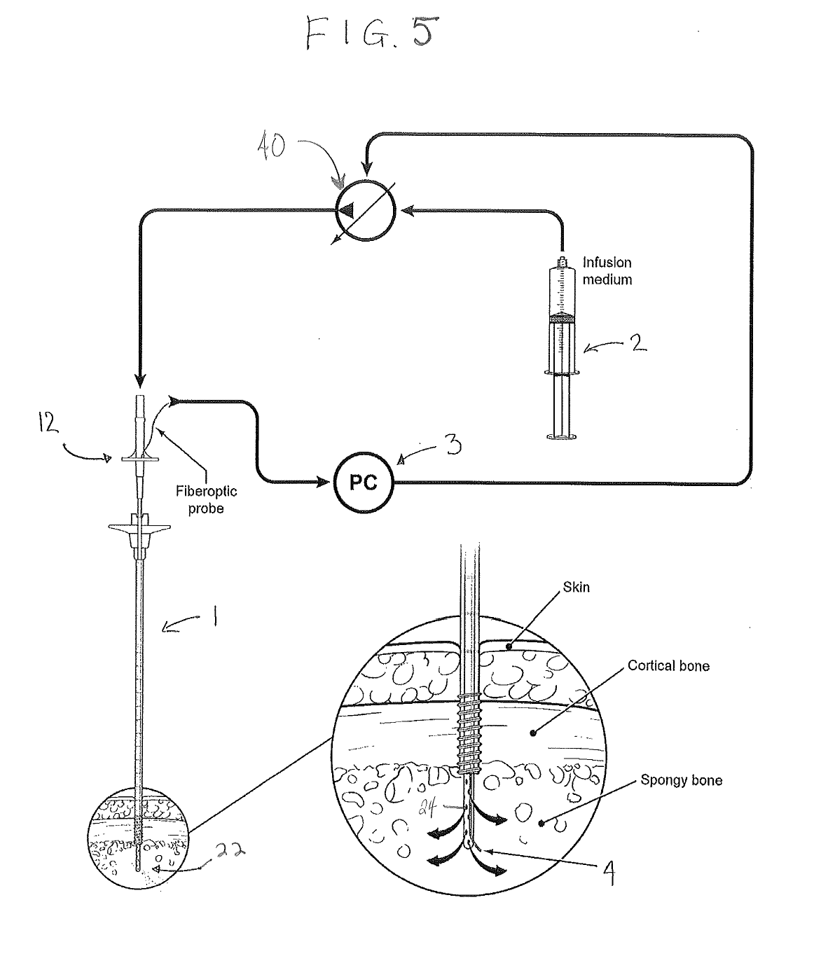

In accordance with some embodiments, (e.g. see FIG. 4a), access to the bone marrow can be provided by the access cannula 6 and a penetrator 7, after which the penetrator 7 can be withdrawn from the access cannula 6 and replaced with the infusion cannula 8.

The dimensions of the access cannula 6, infusion cannula 8, and penetrator 7 are not particularly limited, and can be in accordance with known intra-bone access, infusion, and penetrating devices. In general, the access cannula 6 is sized large enough to provide rigidity necessary for insertion through a patient's skin and into a bone, and to allow for insertion of an infusion cannula 8 sized to infuse an agent at a desired flow rate and a penetrator 7 when used for obtaining access to the bone marrow, while maintaining a small profile to minimize the post-operative care required. The access cannula 6 is at least larger in cross-section than the infusion cannula 8 and penetrator 7 which are insertable therethrough. The length of the access cannula 6 can vary, and is generally of sufficient length to provide access from the surface of the skin to the interior of an underlying bone. The length of the infusion cannula 8 is preferably at least the same length or longer than the length of the access cannula 6. However, it may also be possible to provide an access cannula 6 that is shorter in length than the access cannula 6, as long as one or more agents infused through the access cannula 6 are released onto the bone marrow, and as long as the intra-bone device 1 is adapted with a pressure sensor 4 configured and arranged to monitor intra-bone pressure. The length of the penetrator 7 is preferably longer than the length of the access cannula 6 such that it can pierce the skin and/or bore into the bone.

According to an exemplary embodiment, the access cannula 6 ranges from about 5 gauge to 7 gauge, and the penetrator 7 and infusion cannula 8 range from about 7 gauge to about 9 gauge. For example, the access cannula 6 may be about 6 gauge with an interior lumen large enough to accommodate a penetrator 7 and infusion cannula 8 which are about 8 gauge.

The dimensions of the access cannula 6, penetrator 7 and infusion cannula 8 can vary depending on a variety of factors, such as the location of the target bone. According to exemplary embodiments, as shown in FIGS. 4a-4b, a length d₅ of an elongate portion 31 of an access cannula 6 can range from about 8-20 cm, with a threaded portion 23 provided at a distal end having a length d₆ ranging from about 1-3 cm, a length d₁₀ of an elongate portion 34 of a penetrator 7 can range from about 11-25 cm, a distal end of the penetrator 7 can extend outside of the access cannula 6 by a length d₇ of about 0.5-2.5 cm, a length d₁₁ of an elongate portion 33 of an infusion cannula 8 can range from about 12-26 cm, a distal end of the infusion cannula 8 can extend outside of the access cannula 6 by a length d₇ of about 0.5-2.5 cm. As shown in FIG. 4a, a handle 35 of a penetrator can have a length d₉ of about 6-12 cm, a height d₂ of about 2-6 cm, and a width d₁ of about 1-3 cm. when provided, a hex bolt 19 can be about a 1-2 cm, preferably a 1.5 cm, hex bolt which is disposed within a recess of the handle as shown in FIG. 4a when the handle 35 is connected to the elongate portion 34 of the penetrator 7. The recess may, for example, extend a depth d₃ of about 1-2 cm. The hub 30 of the access cannula 6 is not particularly limited in size, but can, for example, have a height d₄ (excluding the coupling 15) of about 0.7-2 cm.

The distal end 22 of the infusion cannula 8 is adapted for releasing one or more agents therethrough. In particularly preferred embodiments, at least one opening 24 (e.g., see FIG. 4b) is provided at the distal end 22. Any number of openings 24 may be provided at any location, in any size, and in any configuration to provide a desired release profile.

Near the distal end 22 of the infusion cannula 8, a pressure sensor 4 is provided. In embodiments in which the distal end 22 of the infusion cannula 8 does not protrude beyond the distal end 11 of the access cannula 6, the pressure sensor 4 can be provided in an alternate location if desired, such as a location in the access cannula 6 disposed within the bone marrow. The pressure sensor 4 is configured and arranged to constantly monitor intra-bone pressure during a procedure. The pressure sensor 4 is in connection with a mechanism 3 that receives pressure measurements from the pressure sensor 4. The mechanism 3 is in connection with an infusion device 2, such that the mechanism adjusts the infusion of one or more agents into the bone based on the pressure measurements received from the pressure sensor 4. According to preferred embodiments, the pressure measurements are carried out continuously throughout a process, and the infusion adjustments are made as needed so as to maintain intra-bone pressure within a desired range.

According to an exemplary embodiment, the pressure sensor 4 extends from a location near the distal end 22 of the infusion cannula 8 and along the elongate portion 33, and emerges at the proximal end 21 of the infusion cannula 8 (e.g. as shown in FIG. 1b) where it then proceeds to the mechanism 3. According to various embodiments, a recess 5 may be provided in an outer surface of the elongate portion 33 of the infusion cannula 8 for receiving the pressure sensor 4. According to various embodiments, the recess 5 is configured such that the pressure sensor 4 is flush or below the outer surface of the elongate portion 33 to protect the pressure sensor 4 from becoming damaged during insertion and withdrawal of the infusion cannula 8. It is also possible to dispose the pressure sensor 4 along an interior surface of the infusion cannula 8, or embed the pressure sensor 4 within the walls of the infusion cannula 8 with the pressure sensor 4 exposed at a portion that is disposed within the bone marrow when the infusion cannula 8 is positioned within the bone marrow.

As shown in the embodiment of FIG. 4a, access to the bone marrow can be provided by the access cannula 6 and a penetrator 7. As shown, the penetrator 7 can include an elongate portion 34 and a handle 35. The penetrator 7 is insertable through the access cannula 6. Thus, for example, the elongate portion 34 of the penetrator 7 can be inserted through a lumen within the access cannula 6 (much like insertion of the infusion cannula), and the devices can together be inserted through the skin and into the bone. In order to facilitate piercing of the skin and boring into the bone, the penetrator 7 can have a sharpened beveled tip at a distal end 13. According to an exemplary embodiment, the distal end 13 can be threaded like a screw, such as in the form of a drill bit (e.g. as shown in FIG. 4a) with a tapered and fluted tip for facilitating insertion through the skin and bone. According to various embodiments, the penetrator can be grasped by the handle 35 and twisted through the bone as force is applied downward. The device can, thus, be inserted manually or can be power driven to facilitate piercing of the very hard bone structure. The access cannula 6 and/or the penetrator 7 can be provided with depth markings (not shown) on an outer surface to provide a user with a guide as to how far the device must be inserted for proper positioning within the bone.

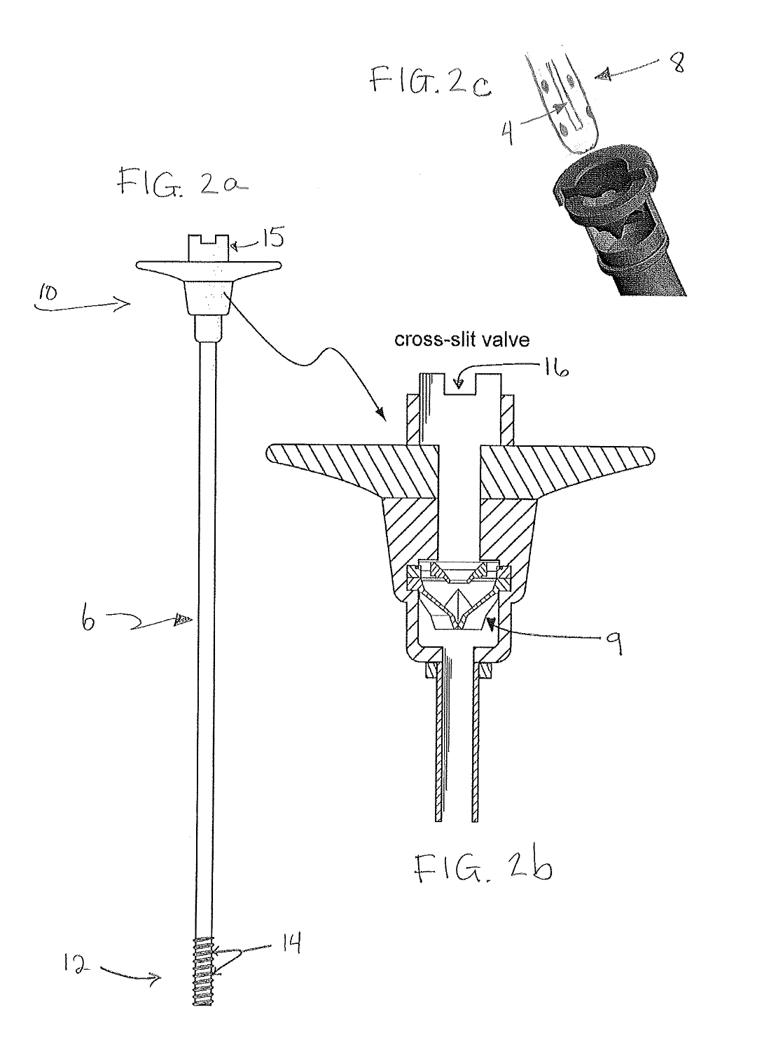

As shown in FIGS. 1a and 2a, threads 14 can further be provided on the distal end 11 of the access cannula 6 to aid in securing the access cannula 6 within the bone during use, thus providing further stability during insertion and withdrawal of various instruments therethrough (e.g. so that the penetrator 7 can be withdrawn from the access cannula 6 and the infusion cannula 8 can be inserted through the access cannula 6 for the subsequent infusion of agents).

As illustrated in FIG. 5, an infusion device 2 can be in connection with the infusion cannula 8 which has been inserted within the access cannula 6. The infusion device 2 can be in any form suitable for providing a controllable flow of agents to the infusion cannula 8. For example, as shown in FIG. 5, the infusion device 2 can generally be in the configuration of a syringe. According to various embodiments, the infusion cannula 8 has a blunt tip (e.g. rounded). The infusion cannula 8 may be provided with or without an opening at its distal end.

According to various embodiments, the infusion device 2 can be in direct connection with the access cannula 6. In such embodiments in which the infusion device 2 is in direct connection with the access cannula 6, the access cannula 6 could be configured for infusion of one or more agents therethrough. For example, the device would gain access to a bone similar to the above method, wherein the penetrator 7, together with the access cannula 6 could be inserted through the skin and into the bone with the distal end 11 of the access cannula 6 positioned within the bone marrow. The penetrator 7 could then be withdrawn from the access cannula 6, and the infusion device 2 attached thereto.

Preferably, the infusion device 2 is in connection with the infusion cannula 8 which is insertable within the access cannula 6, for example, as shown in FIG. 5. As such, once the device is positioned within the bone marrow, with the access cannula 6 and/or infusion cannula 8 disposed within the bone marrow, the infusion device 2 could be attached to the infusion cannula 8 for infusion of one or more agents therethrough.

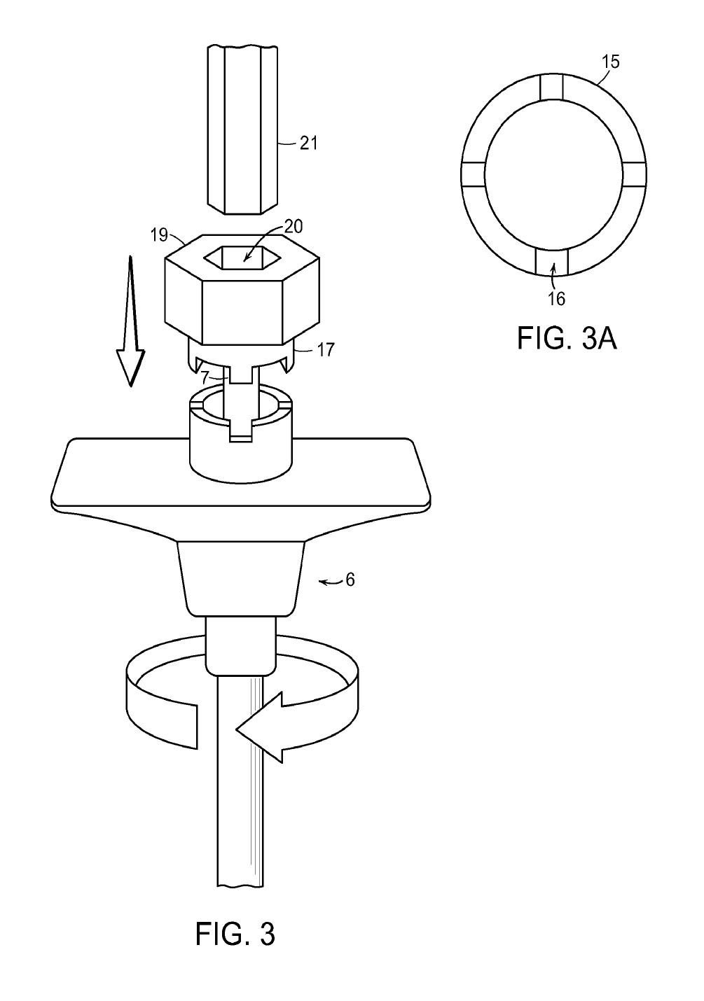

According to various embodiments, the access cannula 6 can be configured so as to provide an interlocking communication with a penetrator 7, an infusion cannula 8 and/or an infusion device 2 when these devices are inserted through an inner lumen of the cannula 6. For example, as shown in FIGS. 2a-3, a proximal end 10 of the access cannula can be provided with a coupling 15 that is shaped to corresponds to one or more mating portions on a proximal end 12 of a penetrator 7, a proximal end 21 of the infusion cannula 8 and/or located in the infusion device 2. As shown in FIGS. 2a-3, one or more notches 16 can be provided in the coupling 15 and one or more corresponding protrusions 17 can be provided in the penetrator 7, infusion cannula 8 and/or infusion device 2. In accordance with an exemplary embodiment, as shown in FIGS. 3-4a, the penetrator 7 is provided with a removable handle 35 that can be attached to the elongate portion 34 through any suitable releasable attachment means. For example, the handle 35 can include a protrusion 41 that mates with a proximal portion of the elongate portion 34 through a suitable coupling 19. As shown in the embodiment of FIGS. 3-4a, the coupling can be a hex bolt with an opening 20 through which the protrusion 41 is insertable. After insertion of the protrusion 41, the coupling (e.g. hex bolt) 19 can be tightened to fix the handle 35 to the elongate portion 34. As shown in FIG. 3, one or more protrusions 17 can be disposed at a side of the coupling 19 and positioned to engage the notches 16 in the coupling 15 disposed on the access cannula 6. Prior to engagement between protrusions 17 and notches 16, the penetrator 7 could be rotatable within the access cannula 6 for suitable placement. After the protrusions 17 and notches 16 are engaged, rotation of the penetrator 7 handle 35 will rotate the entire access system (the penetrator 7 and the access cannula 6).

As shown in FIG. 2c, a valve 9 can be disposed within the hub 30 of the access cannula 6. The valve 9 can be any conventional type of valve, including but not limited to cross-slit valves, one-way valves, two-way valves, etc. The valve provides for a fluid tight seal between the access cannula 6 and the device inserted therein (e.g. penetrator 7, infusion cannula 8, infusion device 2). Further, the valve 7 can, in some embodiments, be in communication with the mechanism 3 that receives pressure readings from the pressure sensor 4. As such, in an embodiment wherein the infusion device 2 is directly inserted within the access cannula 6, the mechanism 3 can be configured to continuously receive intra-bone pressure readings from the sensor 4 and can communicate with the valve 7 so as to adjust flow of agent therethrough and into the bone marrow.

As shown in FIG. 4b, an infusion cannula 8 is inserted within the access cannula 6 with the distal end 22 of the infusion cannula 8 extending beyond the distal end 11 of the access cannula 6. The pressure sensor 4 is in the form of a fiberoptic probe that extends from a location near the distal end 22 of the infusion cannula 8 to the proximal end 21. As shown, the pressure sensor 4 can extend from the handle 32 of the infusion cannula 8 to a mechanism 3 that receives pressure readings. As shown, the infusion cannula 8 can be provided with one or more depth markers 22 on an outer surface to provide a user with guidance as to the depth to which the infusion cannula 8 should be inserted so as to be positioned appropriately within the bone.

FIG. 5 shows a schematic of the intra-bone device 1, which includes the access cannula 6 and infusion cannula 8. As shown, the intra-bone device is inserted in the bone, with the distal end 11 of the access cannula 6 extending through the cortical bone and into the spongy bone, and the distal end 22 of the infusion cannula 8 extending into the spongy bone. As the infusion device 2 provides one or more agents to the infusion cannula 8, the one or more agents pass through the infusion cannula 8 and into the bone through the openings 24 in the distal end 22. Pressure sensor 4 continuously takes readings of intra-bone pressure during the infusion, and transmits these readings to the mechanism 3. Based on pre-determined allowable pressure levels that are input in the mechanism 3, the mechanism then transmits instructions to maintain, reduce, or increase the infusion so as to maintain intra-bone pressure within the pre-determined pressure levels.

Preferably, intra-bone pressure is maintained at levels not exceeding systemic blood pressure. It is understood my one skilled in the art that blood pressure drops during anesthesia. Accordingly, in certain embodiments, intra-bone pressure is maintained at levels not exceeding 40-50 mmHg, preferably at levels not exceeding 30-40 mmHg, preferably at levels not exceeding 20-30 mmHg, and preferably at levels not exceeding 25-30 mmHg.

The mechanism 3 can be in the form of any conventional control mechanism, computer, monitor, etc. that is adapted for receiving data from the pressure sensor 4, comparing the data to a pre-determined allowable range that is input, and communicating instructions to stop, slow, maintain or increase the infusion of one or more agents based on the comparing. According to an embodiment as shown in FIG. 5, the mechanism 3 is in communication with a controller 40 that adjusts the infusion of one or more agents from the infusion device 2. The controller 40 can, in some embodiments, be a valve or similar mechanism that can be completely closed to stop infusion, and can be opened at variable amounts to allow for increased and decreased flow from the infusion device 2 to the infusion cannula 8. According to other embodiments, the infusion device 2 may have controller thereon (not shown) that receives input from the mechanism 3 and that adjusts the rate at which agents are infused from the infusion device 2. While the infusion device 2 is shown in the form of a syringe, any other conventional infusion device can be used to provide a flow of agents to the infusion cannula 8.

According to an exemplary embodiment, a method is provided for performing a bone marrow transplant using the apparatus described herein. In particular, an intra-bone device 1 is inserted into a target bone with a proximal portion of the device being disposed in or near the bone marrow. In particular, a penetrator 7 can be inserted within an access cannula 6 until protrusions 17 on the penetrator 7 couple with the coupling 15 on the access cannula 6. The penetrator 7 and access cannula 6 can then together be advanced through the skin of a patient and into a target bone. In certain embodiments, the distal end 13 of the penetrator 7 has a sharpened tapered and fluted tip that extends proximally into a threaded portion 23. As such, the entire device might be twisted through the bone as force is applied downward. In some embodiments, the device can be power driven into the bone. If depth markings are provided on the access cannula 6 and/or the penetrator 7, the user can insert the device until the desired depth is reached indicating that the bone marrow has been reached. The penetrator 7 is then removed from the access cannula 6 and the infusion cannula 8 is inserted in its place. In some embodiments, an infusion cannula 8 can be eliminated and an infusion device 2 can be directly attached to the access cannula. In such a situation, a pressure sensor 4 may be provided at a distal end 11 of the access cannula 6. In embodiments in which the infusion cannula 8 is inserted through the access cannula 6, the infusion cannula 8 is preferably inserted until its distal end 22 extends outside of the distal end 11 of the access cannula 6. A pressure sensor 4 may further be provided at the distal end of the assembly (either on the distal end 11 of the access cannula 6 or the distal end 22 of the access cannula 6). As shown in FIG. 5, the pressure sensor 4 is provided at the distal end 22 of the infusion cannula 8. The pressure sensor 4 is in communication with the mechanism 3 which, in turn, is in communication with the infusion device 2 itself or a fluid line from the infusion device to the infusion cannula 8.

The infusion device 2 is placed in communication with the infusion cannula 8. As one or more agents are infused into the bone marrow through the infusion cannula (e.g. openings 24), the pressure sensor 4 constantly measures intra-bone pressure and transmits these readings to a mechanism 3 in communication with the pressure sensor 4. The transmitted pressure readings are compared by the mechanism 3 with a pre-determined pressure range. If the pressure readings are higher than desired, then the mechanism 3 transmits a signal to reduce or stop the infusion of agents. For example, the mechanism 3 can transmit a signal to a controller or valve 40 to reduce or stop the infusion of agents. If the pressure readings are acceptable, then the infusion can continue without modification. If the pressure reading is lower than desired, the mechanism 3 can transmit a signal to a controller or valve 40 to increase the infusion of agents. According to an exemplary embodiment, the mechanism is configured to maintain intra-bone pressure at levels not exceeding systemic blood pressure.

The one or more agents delivered to the bone can be any agent. Exemplary agents include, for example, prodrugs, diagnostic agents, imaging agents, therapeutic agents, chemotherapeutic agents, pharmaceutical agents, drugs, synthetic organic molecules, proteins and peptides. According to various embodiments, the one or more agents delivered to the bone can include, but are not limited to, bone marrow, hematopoetic stem cells, mesenchymal stem cells and gene therapy vectors.

In one embodiment, the agent is a chemotherapeutic agent. the term “chemotherapeutic agent” is meant to include a compound or molecule that can be used to treat or prevent a cancer. A “chemotherapeutic agent” is meant to include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

According to various embodiments, the one or more agents can be delivered to the bone to treat a variety of cancers. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

In certain embodiments, the cancers include, but are not limited to, acute leukemias, brain tumors, breast cancer, Hodgkin's disease, multiple myeloma, neuroblastoma, non-Hodgkin's lymphomas, ovarian cancer, sarcoma and testicular cancer.

In certain exemplary embodiments, the methods of the invention are used to deliver intrathecal chemotherapy at a slow and constant rate under low pressure (i.e. pressure that does not exceed systemic blood pressure).

The methods of the present invention are also used to deliver anesthesia. In particular, embodiments, the methods are used in epidural anesthesia when you need a steady low flow of anesthesia delivered.

The present invention also relates to kits and methods involving such intra-bone access and infusion devices.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Optimization of Intrabone Delivery of Hematopoietic Progenitory Cells in a Swine Model Using Cell Radiolabelling

There is limited investigation of intrabone (IB) hematopoietic cell transplantation in small mammals and human trials. It is not known in humans whether hematopoietic progenitor cells (HPCs) injected IB are immediately retained in the bone marrow or rapidly enter into venous circulation before eventually homing to the marrow. The ability to track HPCs in vivo provides insight into their distribution following injection. An optimized method for direct-IB infusion of HPCs in humans, which maximizes cellular retention in the bone marrow, has not yet been developed. Traditionally, [111]indium (¹¹¹In) oxine has been used to label hematopoietic cells; however, this requires a high dose of radiation exposure to the labeled cells, reducing viability. Moreover, images are suboptimal because ¹¹¹In is a single photon emitter that is detected with single photon emission computed tomography (SPECT) cameras, which have intrinsic limitations in resolution and sensitivity. An alternative to single photon emitters are positron emitters that emit two oppositely directed gamma rays after annihilation with an electron. PET has inherently higher sensitivity (up to ten fold) and spatial resolution than SPECT enabling lower doses of radioactivity to be used without compromising the quantitation of PET activity. Thus PET cell imaging techniques were applied using the long-lived PET emitter ⁸⁹Zr (Sato, et al), to visualize HPCs in this study. Using human CD34+ HPCs, presented herein is a method of radionuclide labeling, using a long-lived (half-life 78.4 h) positron emitter [89]zirconium (⁸⁹Zr), comprising of simple mixture with protamine sulfate that results in cellular retention of small quantities of radioactivity that is non-transferable between cells. The uptake activity is capable of detection by positron emission tomography (PET) with high sensitivity and high spatial resolution when fused with computed tomography (CT), over time. Using domestic swine, the experiments detailed herein show that high intra-marrow pressure, due to either high volume or high flow-rate, led to extravasation into surrounding muscle and rapid drainage into the central venous circulation. By optimizing the needle design, and by carefully controlled volume and infusion rate with real-time intramarrow pressure monitoring, a high retention of HPCs in the marrow can be achieved using a volume of 10 ml and a flow rate <0.2 ml/s injected into a single site in the hemipelvis.

Porcine Pelvic Vascular Supply and Drainage Following IB Injection

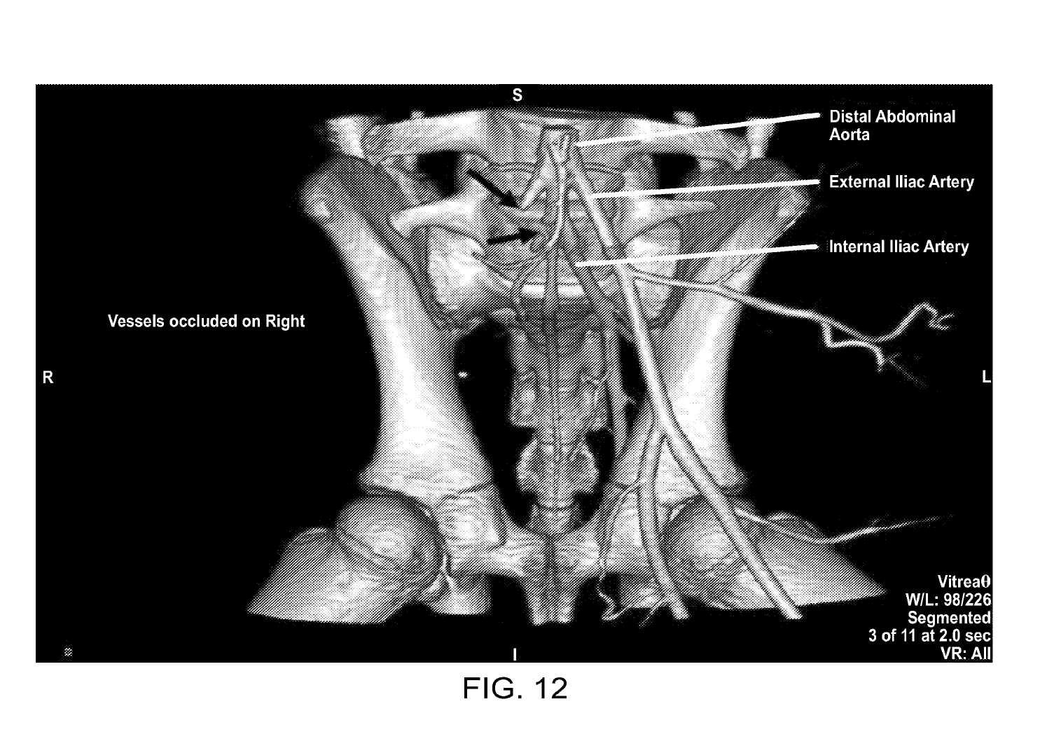

Utilizing selective catheterization and 3D CT angiography, the internal iliac artery was identified as the main supply of blood into the pelvic bone marrow. Unlike human anatomy, the external and internal iliac arteries in the pig arise as terminal branches of the abdominal aorta rather than arising from a common iliac trunk (FIG. 12). Venous drainage of the iliac bone was into short veins which eventually drained into a common iliac vein. Following IB injection, contrast was observed first in the pelvic bone marrow followed by opacification of short pelvic veins and common iliac vein. When the common iliac vein was occluded, contrast drainage from the pelvic bone marrow occurred via veins along the iliac crest into the lumbar venous plexus, and via cross-pelvic collaterals (and uterine veins in female pigs) to the contralateral common iliac vein.

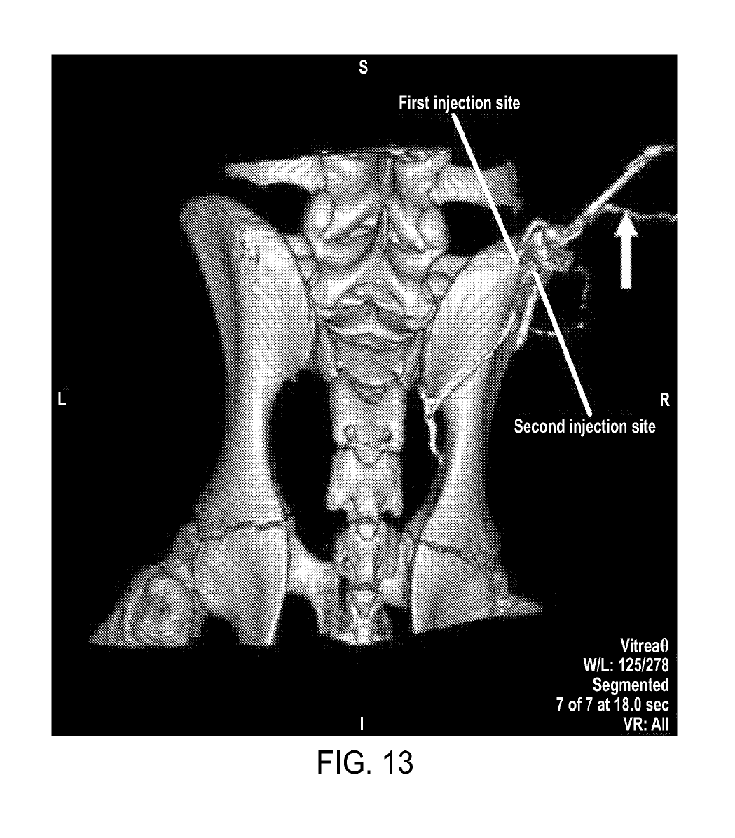

Contrast injection into the bone marrow at rates exceeding 0.2 ml/s, volumes >10 ml and injections performed by hand, all resulted in extrabone contrast leakage at the site of injection. When more than one IB access was made in the ipsilateral pelvis, contrast was observed to leak through the non-injected site regardless of the infusion rate and the distance between the two sites (FIG. 13).

Hemodynamic Changes During IB Injection

Lower injection volumes and slower injection rates produced small increases in intramarrow pressure that were just above systemic arterial pressure and were accompanied by a minimal increase in post-injection peak pulmonary artery systolic pressure (PASP) (Table 1, shown below).

TABLE 1
Peak IB pressure and change in pulmonary artery systolic pressure
following IB injection at varying rates and volumes.

| | | Number | Δ Intrabone | Δ Peak Pulmonary |
|---|---|---|---|---|
| Volume | Rate | of | Marrow | Artery Systolic |
| injected | injected | Exper- | Pressure (mmHg) | Pressure (PASP) |
| (mL) | (mL/s) | iments | Mean ± S.E. | (mmHg) Mean ± S.E. |
| 10 | 0.1 | 4 | 159.3 ± 57.7 | 1.5 ± 0.7 |
| 10 | 0.2 | 3 | 300.3 ± 149.3 | 3.5 ± 1.0 |
| 10 | 0.5 | 2 | 803.4 ± 483.0 | 13.0 ± 5.1 |
| 10 | 1.0 | 2 | 1196.0 ± 265.5 | 8.9 ± 9.3 |
| 10 | 2.5 | 3 | 1689.0 ± 116.0 | 2.2 ± 1.0 |
| 5 | 0.1 | 2 | 128.7 ± 60.2 | −0.5 ± 0.4 |
| 5 | 0.2 | 2 | 256.7 ± 42.7 | 1.3 ± 1.3 |
| 5 | 0.5 | 1 | 802.9 | 6.1 |
| 5 | 1.0 | 1 | 1490.8 | 0 |
| 5 | 2.5 | 1 | 997.5 | 2 |

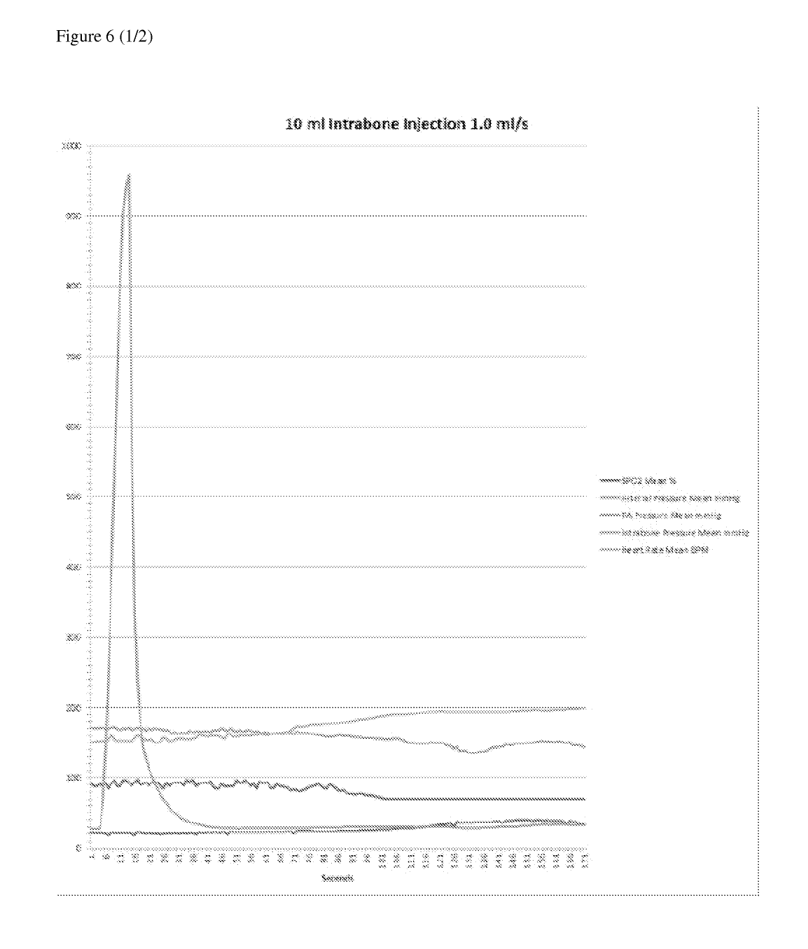

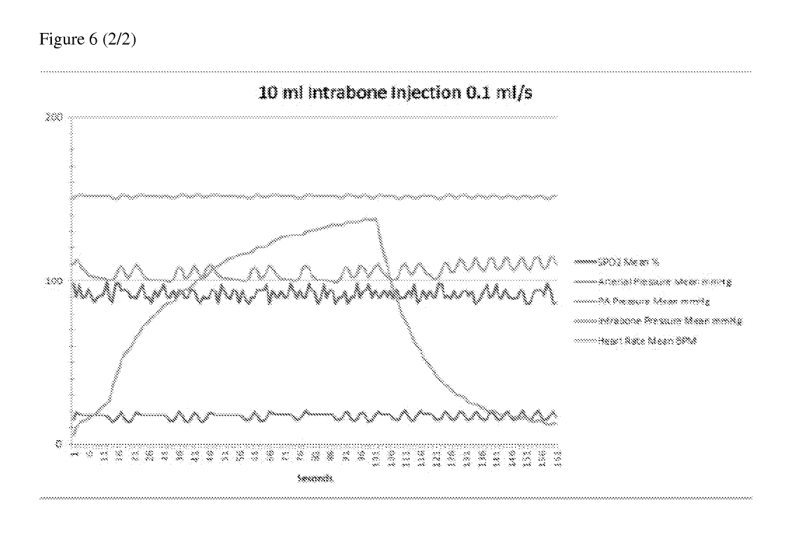

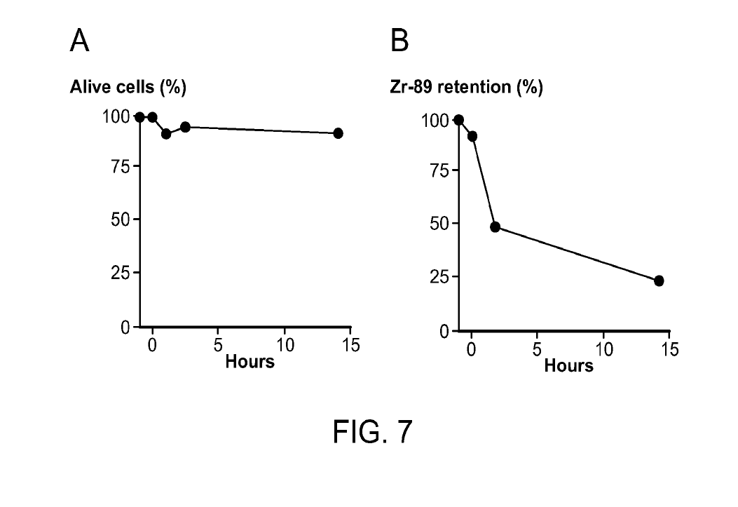

Larger injection volumes and more rapid injection rates were associated with markedly elevated intramarrow pressures and greater increases in post-injection PASP (Table 1 and FIG. 6). At larger volumes and more rapid injection rates, the change in post-injection PASP appears to decrease over time, however because volumes and rates were sequential in each animal, baseline PASP remained elevated after injections exceeding rates of 1 ml/s resulting in lower net difference. The overall clinical status of animals after high volume/high injection rate injections, which included systemic hypotension, tachycardia, hypoxemia and increased PASP suggested pulmonary embolism occurred after more rapid or larger volume IB injections and this was confirmed by CT angiography in several cases (data not shown).

Cell Labeling

hCD34+ cells were labeled with ⁸⁹Zr up to 7.68 μCi/million cells. At this dose, hCD34+ cells survived well up to the 14 h observation period in vitro (FIG. 7A). This assured that the 89Zr cell labeling procedure did not negatively affect the longevity of hCD34+ cells survival. Release of some 89Zr from the labeled cells was observed over time (FIG. 7B), which could result in a safety mechanism to minimize unnecessary radio-exposure to the cells after imaging has finished.

Injection of ⁸⁹Zr-Df IV or IB

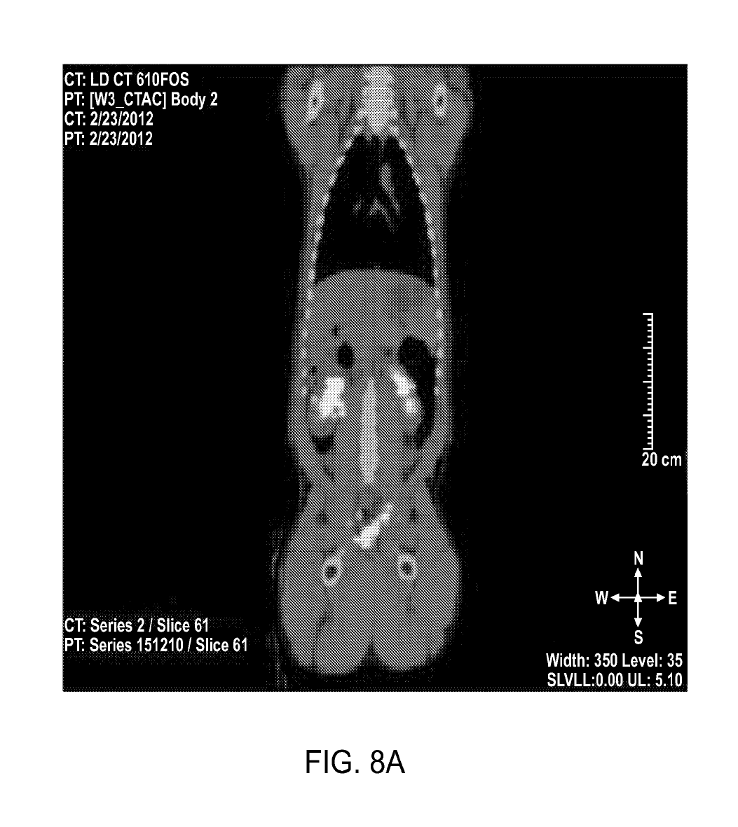

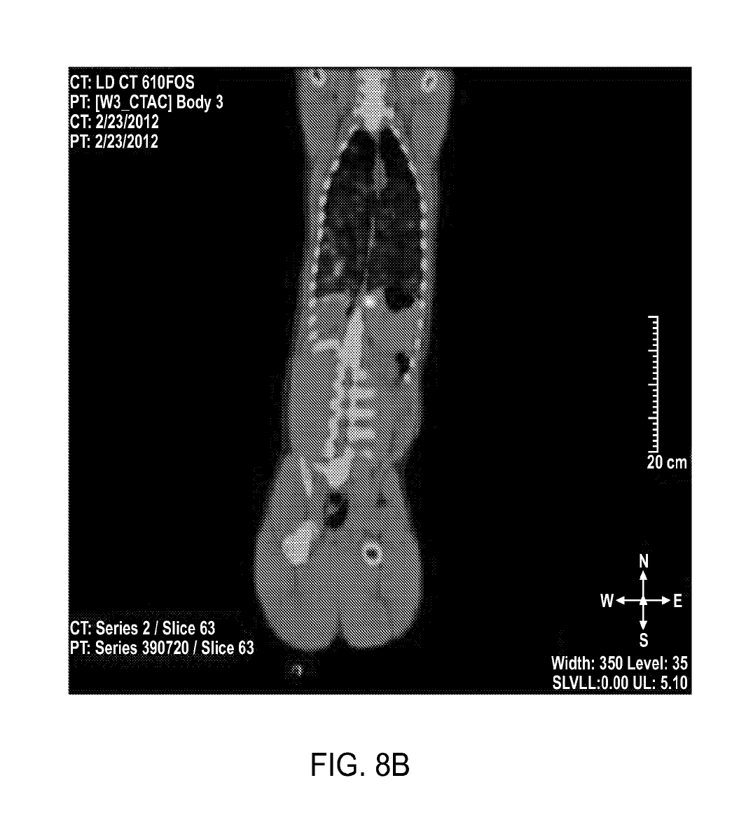

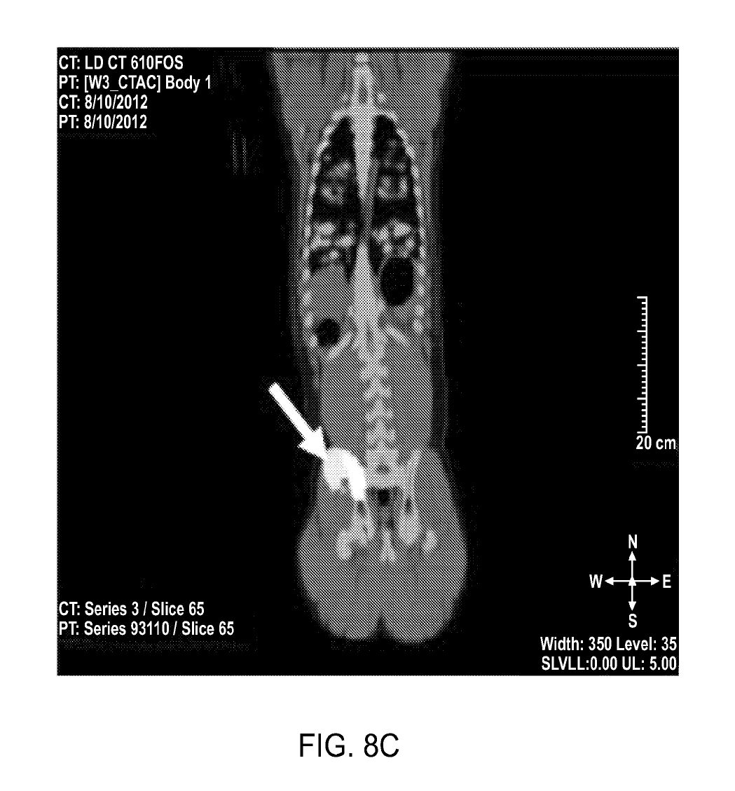

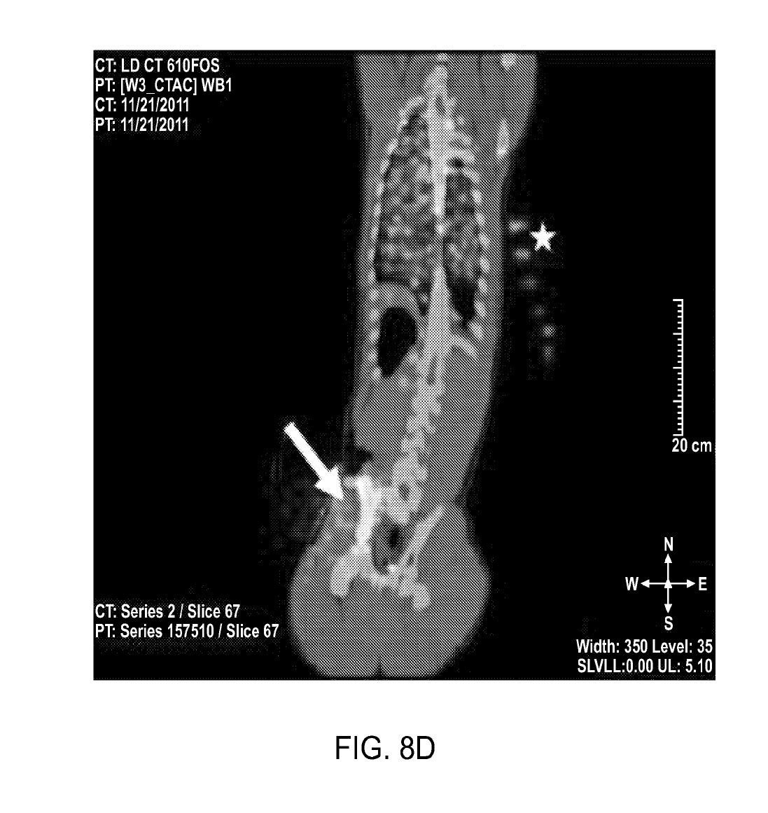

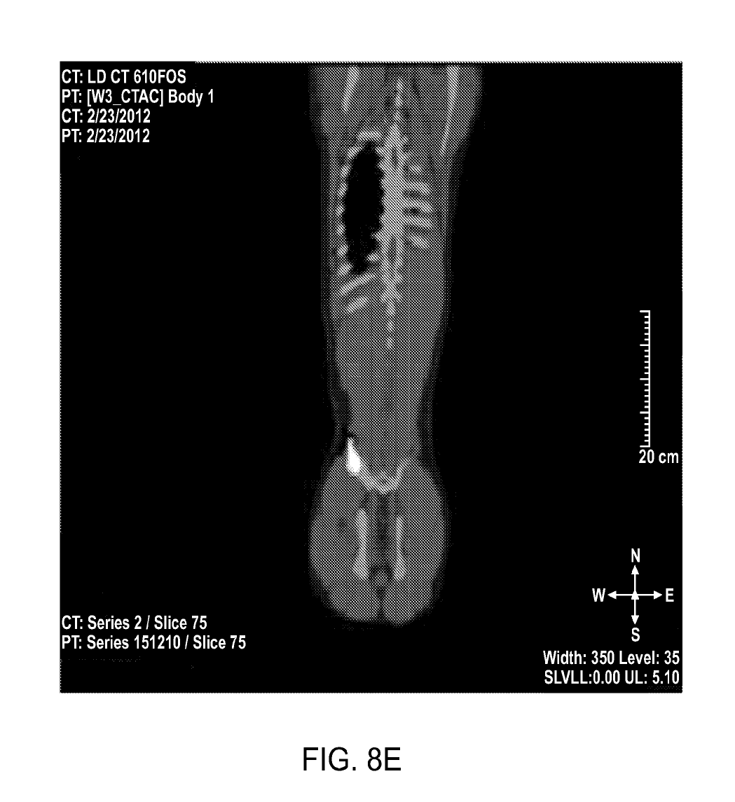

Following injection of ⁸⁹Zr-Df either IV or IB, measurements were obtained of the radioactivity in and around the iliac bone and lungs including the VOI maximum and mean pixel value, VOI in ml, total radioactivity in counts, and % ID after the first hour. Following IV or IB ⁸⁹Zr-Df administration without cells, the agent was largely confined to the blood pool with little or no lung radioactivity within the first hour and in the case of IB injections, radioactivity was demonstrated at the site of injection (33.5% ID) and within the renal collecting system, ureters and bladder (66.5% ID) (Table 2, shown below, and FIG. 8A). There was no retention of ⁸⁹Zr-Df in the lungs in either case.

TABLE 2
The region of interest (ROI) maximum and mean pixel value, ROI volume, total radioactivity in counts, and percent of injected dose
(% ID) after the first hour within the pelvic bone (intra-pelvic bone); outside the pelvic bone but within the pelvis (extra-pelvic); and in the
lungs; for each pig administered ⁸⁹Zr-deferoxamine chelate (⁸⁹Zr—Df) or ⁸⁹Zr-radiolabeled human CD34+ cells (⁸⁹Zr—hCD34+) either IV or IB.

| | | | | | Intra- | Intra- | Volume | Extra- | Extra- | Volume |
|---|---|---|---|---|---|---|---|---|---|---|
| | | | | | pelvic | pelvic | of intra- | pelvic | pelvic | of extra- |
| | | | | Volume | bone | bone | pelvic | bone | bone | pelvic |
| | Weight | | | injected | VOI | VOI | bone | VOI | VOI | bone |
| [symbol] ID # | (kg) | Injection | Group | (ml) | (Max) | (Mean) | VOI (ml) | (Max) | (Mean) | VOI (ml) |
| 32590 | 52 | [symbol] | Control | [symbol] 0 | 338.9 | 314.42 | 9.49 | 91.45 | 15.32 | 132.26 |
| 32592 | 5 [symbol] | ⁸⁹Zr— | IV | [symbol] 0 | 0 | 0 | 0 | 1. [symbol] | 0.17 | 27.52 |
| | | hCD34+ | | | | | | | | |
| 34489 | 43 | ⁸⁹Zr— | IB-hand | 5 ( [symbol] ) | 303.23 | 174.38 | 5 | [symbol] .99 | 2 [symbol] 3 | [symbol] .03 |
| | | hCD34+ | (2 | | | | | | | |
| | | | [symbol] | | | | | | | |
| | | | [symbol] | | | | | | | |
| 34490 | 43 | ⁸⁹Zr— | IB-hand | [symbol] 0 | [symbol] 74.28 | [symbol] 72.53 | [symbol] .41 | 293.04 | 150. [symbol] 5 | 9.41 |
| | | hCD34+ | | | | | | | | |
| 331 [symbol] 5 | 47 | ⁸⁹Zr— | IB-hand | [symbol] 0 | 98.52 | 3 [symbol] | 9.08 | 15.13 | 5.32 | 5.35 |
| | | hCD34+ | | | | | | | | |
| 31717 | 51 | ⁸⁹Zr— | IB-hand | [symbol] 0 | 1114.79 | 231.79 | [symbol] | [symbol] 5 | 113.69 | [symbol] 7.04 |
| | | hCD34+ | | | | | | | | |
| 3 [symbol] 85 | 44 | ⁸⁹Zr— | IB- [symbol] | [symbol] 0 | 27 [symbol] .65 | [symbol] | 4.13 | 15.21 | 5.53 | 2.16 |
| | | hCD34+ | | | | | | | | |
| 331 [symbol] | 52 | ⁸⁹Zr— | IB- [symbol] | [symbol] 0 | 1685.55 | [symbol] 44.9 | 6.3 | 4 [symbol] | 1 [symbol] .57 | 1.62 |
| | | hCD34+ | | | | | | | | |
| 33190 | 50 | ⁸⁹Zr— | IB- [symbol] | [symbol] 0 | 1210.75 | 230.51 | 6.8 [symbol] | 175.87 | 34.27 | 9.95 |
| | | hCD34+ | | | | | | | | |
| 33296 | 40 | ⁸⁹Zr— | IB- [symbol] | [symbol] 0 | 986.39 | [symbol] | 4.93 | 13. [symbol] 3 | 3. [symbol] 5 | 1.6 |
| | | hCD34+ | | | | | | | | |
| 32261 | 4 [symbol] .6 | ⁸⁹Zr— | IB- [symbol] | [symbol] 0 | [symbol] | 95 [symbol] .73 | 2.54 | [symbol] 2 | 25.57 | 1.45 |
| | | hCD34+ | | | | | | | | |
| 3 [symbol] | [symbol] 8 | ⁸⁹Zr— | IB- [symbol] | [symbol] 0 | 1145.85 | 200.71 | 5.11 | 40.77 | 13.41 | 3.31 |
| | | hCD34+ | | | | | | | | |
| | | | | | Intra- | | | | | |
| | | | | | pelvic | Extra- | | Intra- | | |
| | | Lung | Lung | Volume | bone | pelvic | Lung | pelvic | Extra- | |
| | [symbol] | VOI | VOI | of lung | total | total | total | bone | pelvic | Lung |
| | ID # | (Max) | (Mean) | VOI(ml) | radioactivity | radioactivity | radioactivity | % ID | % ID | % ID |
| | 32590 | 2. [symbol] 9 | 0 | 2159.2 [symbol] | 1085.85 | 235 [symbol] | 0 | 33.5% | [symbol] .5% | 0.0% |
| | 32592 | [symbol] 7.21 | 0.38 | 3556.15 | 0 | 4.73 | [symbol] 351.34 | 0.0% | 0.3% | 3 [symbol] % |
| | 34489 | [symbol] .04 | 1.28 | [symbol] 4 | 872. [symbol] 0 | [symbol] 814.27 | 2168. [symbol] 4 | 18.0% | 37.4% | 44.7% |
| | 34490 | 303.32 | 22.29 | 2154.72 | 5017. [symbol] | 1414.79 | 480 [symbol] 8.73 | 9.2% | 2.6% | [symbol] % |
| | 331 [symbol] 5 | 10.8 | 0.0 [symbol] | 3774.47 | 15 [symbol] .18 | 1 [symbol] 2 | 339.70 | 29.7% | 3.5% | [symbol] 5.8% |
| | 31717 | 24.62 | 1.95 | 2852.3 | 2030.4 [symbol] | 3074.1 [symbol] | 5581.49 | 19.0% | 2 [symbol] % | 5 [symbol] .2% |
| | 3 [symbol] 85 | 0 | 0 | 3384. [symbol] 4 | 272.25 | 34.30 | 0 | 95.1% | 4.9% | 0.0% |
| | 331 [symbol] | 0 | 0 | 2949.4 [symbol] | 1542.87 | 25 [symbol] 22 | 0 | 9 [symbol] .4% | 1. [symbol] % | 0.0% |
| | 33190 | 0 | 0 | 14 [symbol] .73 | 1588.21 | 541.02 | 0 | 81.3% | 12.7% | 0.0% |
| | 33296 | 0 | 0 | 2245 | 829. [symbol] 2 | 6.1 [symbol] | 0 | 99.3% | [symbol] .7% | 0.0% |
| | 32261 | 0 | 0 | 2120.79 | 2435. [symbol] 7 | 3 [symbol] | 0 | 98.5% | 1.5% | 0.0% |
| | 3 [symbol] | 0 | 0 | 2087.17 | 1025.63 | 44.39 | 0 | 95.9% | 4.1% | 0.0% |

[symbol]

indicates data missing or illegible when filed

Injection of 89Zr-hCD34+ Cells IV and IB

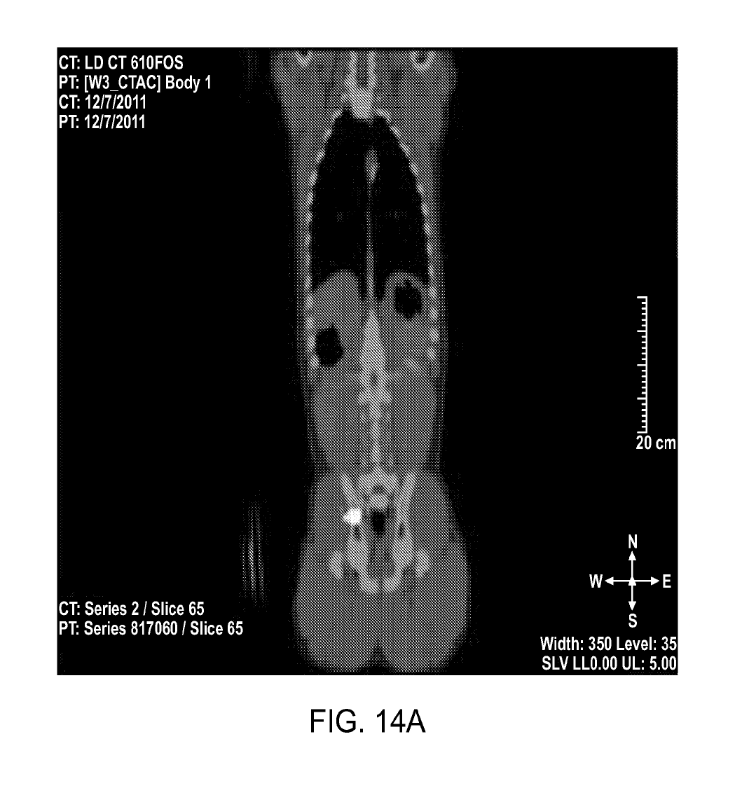

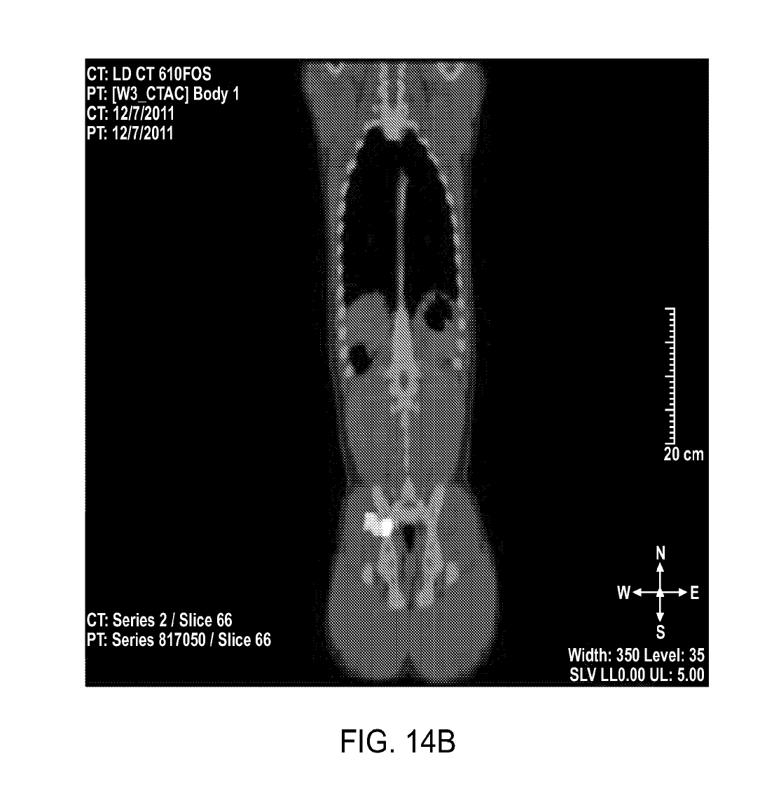

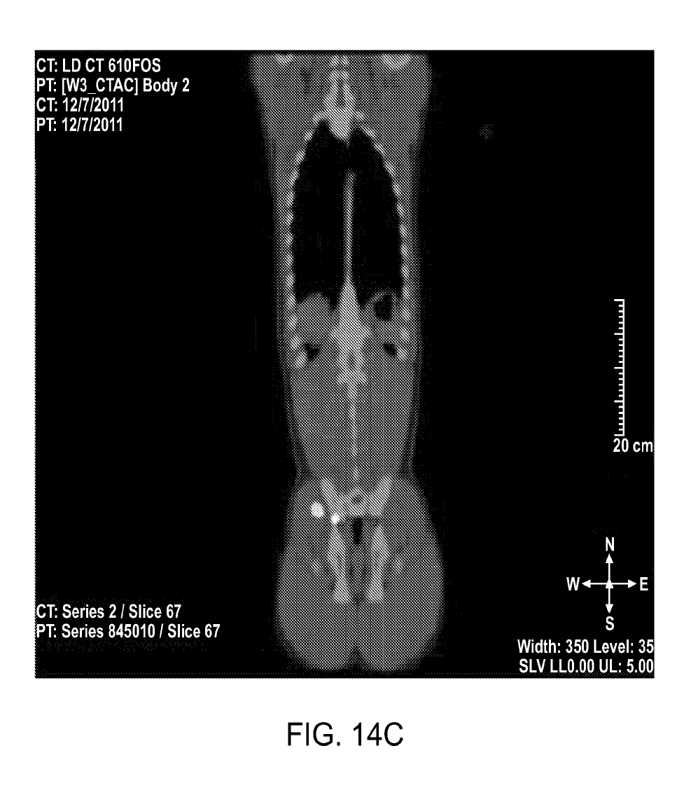

In contrast, following IV ⁸⁹Zr-hCD34+ cell infusion, nearly all of the radioactivity (99.7% ID) was retained in the lungs, demonstrating cell trapping within the alveolar capillaries (Table 2, FIG. 8B). After selective internal iliac artery 89Zr-hCD34+ cell infusion (n=1) radioactivity remained in the pelvic bone however after tracking the radioactivity at 1 hour, radioactivity was shown to advance in the direction of arterial flow beyond the pelvic bone (FIG. 14) and was not clearly retained in the bone marrow.

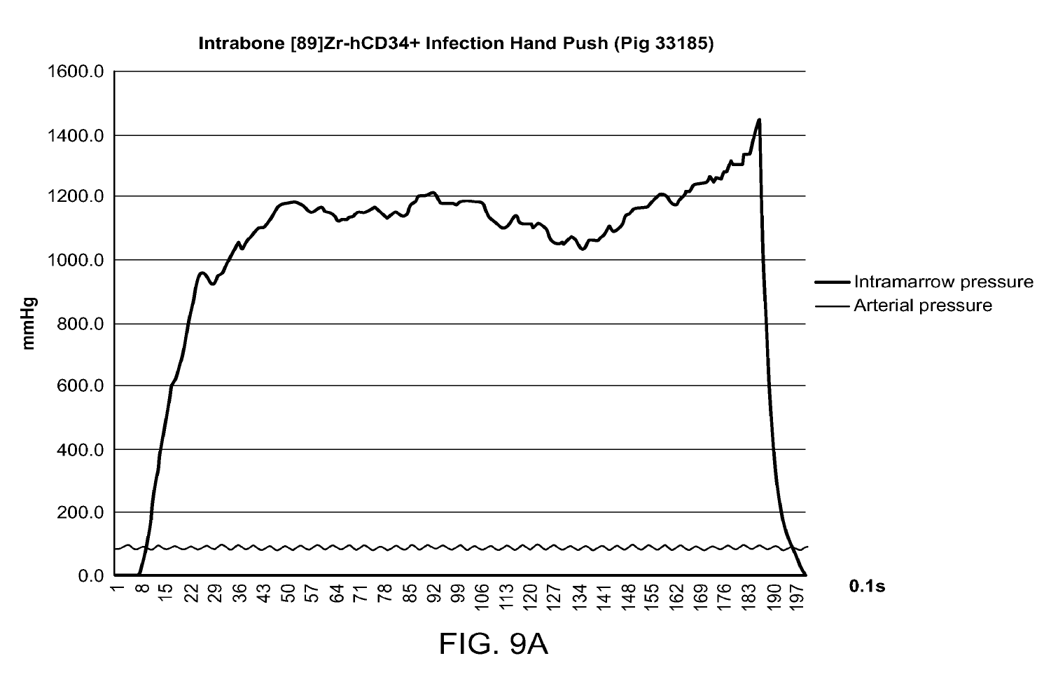

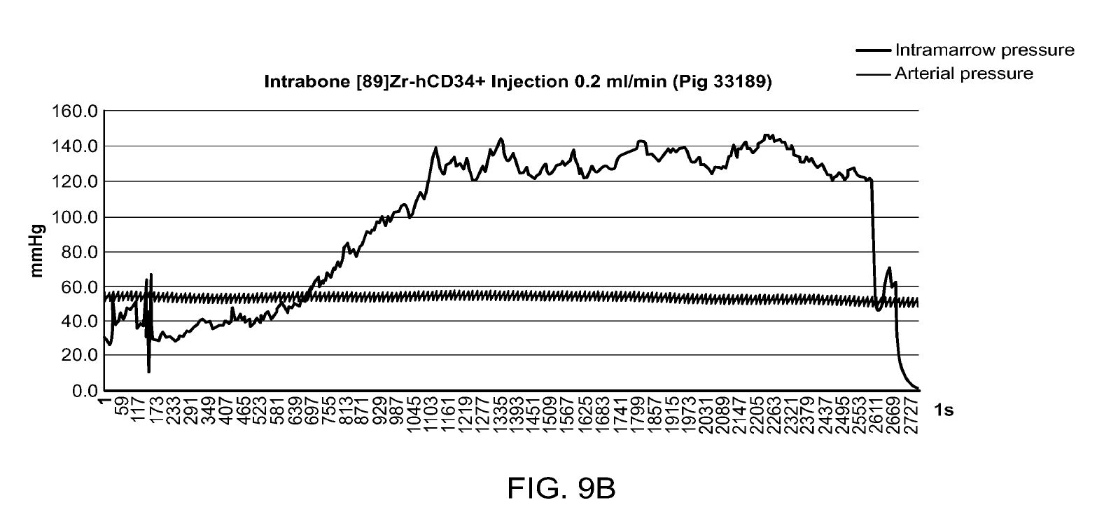

Following IB 89Zr-hCD34+ cell hand-injection into two sites on the ipsilateral pelvis, a significant proportion of radioactivity was demonstrated in the lungs (44.7% ID) suggesting leakage into the venous circulation with lung trapping and outside of the pelvic bone (37.4% ID) due to leakage from the other defect in the bone (FIG. 8C). Following IB ⁸⁹Zr-hCD34+ cell hand-injection via a single site, all animals (n=3) demonstrated some retention of radioactivity in the pelvic bone however there was consistent demonstration of radioactivity within the lungs (52.2-88.2% ID) (Table 2, FIG. 8D). This is consistent with rapid leakage into the circulation with retention in the lungs. As similarly demonstrated with high rate IB contrast injection, hand injection of ⁸⁹Zr-hCD34+ cells generated extremely high intramarrow pressure compared with slow IB infusion (0.2 ml/min) (FIG. 9). Interestingly, when the rate of ⁸⁹Zr-hCD34+ cell injection was decreased to 0.2 ml/min at a single IB site, radioactivity was completely confined to the bone marrow and there was no lung radioactivity at 1 h (n=6) (Table 2, FIG. 8E), with the majority of radioactivity (82.3-99.3% ID) remaining within the pelvic bone consistent with retention of cells within the marrow cavity following IB injection.

Allogeneic HCT is Curative for Many Malignant and Non-Malignant Hematological Conditions.

Routinely, allogeneic HPCs are infused into the recipient IV. For donor engraftment to occur, these allogeneic HPCs are required to migrate or home to the bone marrow niche utilizing cytokine gradients and endothelial cell adhesion molecules to attach to bone marrow endothelium and subsequently transmigrate into the extracellular space where they receive survival and proliferation signals². Although this process is quite successful, there are hurdles that must be overcome, including the pulmonary first-pass effect, whereby the majority of administered HPCs are initially trapped in the pulmonary vasculature³. While not a major impediment to successful adult donor allogeneic HCT where large numbers of HPCs are involved, the use of alternative donor sources such as umbilical cord blood (UCB) derived HPCs is becoming more commonplace in situations where recipients do not have a matched related or unrelated donor. The threshold CD34+ UCB dose for successful engraftment is 1.7×10⁵ cells per kilogram recipient weight4, which is greater than one log less than the average target of 5×10⁶ in HCT from adult donor mobilized peripheral blood. This lower cell dose is one of the factors contributing to delayed donor engraftment resulting in an increased risk of infection and graft failure, which results in increased mortality compared with conventional HCT, and this greatly limits the wider applicability of UCB. This has led investigators to employ the use of dual UCB units in the HCT of adult recipients^{5,6}. Furthermore, UCB HPCs have an inherently decreased homing efficiency compared to their adult counterparts owing to decreased surface CD44 fucosylation7 which is the high affinity E-selectin ligand required for the initial tethering of the HPC to the bone marrow endothelium and is a critical step in homing to the marrow niche. An alternative approach to improve the success of HCT with low HPC numbers may involve bypassing the pulmonary first-pass attrition and directly inject donor HPCs into the recipient bone marrow.

The current prevailing technique for IB injection of UCB in humans involves hand injection (IB push) of 5 ml aliquots through a standard 14 gauge Jamshidi needle into two separate sites on the ipsilateral iliac crest, repeated bilaterally for a total of 20 ml⁸. Our results clearly show that multiple punctures in the pelvic bone lead to intramuscular leakage upon the second injection thus, negating the potential benefits of direct IB injection. Nonetheless, successes have been reported in human trials. The first reported case of successful allogeneic bone marrow transplantation to cure bone marrow failure employed direct injection of donor marrow into the recipient sternal marrow⁹. Frassoni et al. successfully demonstrated single UCB donor engraftment in adult recipients utilizing this IB concept⁸, however a clinically-meaningful improvement in time to donor neutrophil engraftment was not clearly established. Other attempts at IB HCT employing larger volumes did not demonstrate any advantage^{10,11}. Hägglund et al. reported no difference in outcomes between the IV and IB route in allogeneic bone marrow transplantation. Bone marrow was infused IB in a volume of approximately 1000 ml with 99mTc labeled cells and radioactivity was identified immediately in the heart following injection¹⁰. Brunstein et al. also reported on their findings of IB UCB HCT and found no advantage to the IB route in achieving unit dominance compared to the IV route in the double UCB setting¹¹.

These clinical studies do not reflect the experience of superior engraftment following IB transplantation in small mammals¹²⁻¹⁴. Xenotransplantation of human UCB HPCs into mice utilizing direct-IB injection assures long-term engraftment and the ability for secondary SCID mouse repopulation^{15,16}. It is clear that these pre-clinical models cannot predict engraftment kinetics in human clinical trials. Bone marrow blood flow, cardiovascular physiology and anatomical size are not comparable between mouse and man. In humans, the IB route of crystalloid infusion can rapidly restore circulating volume and blood pressure in hypovolemic shock. In fact, in large mammal models it is shown that IB infusion can achieve peripheral to central circulation transit times comparable to those achieved by the IV route^{17,18}. Additionally, there is a substantial risk of fat embolism following the IB infusion route^{19,20}. Furthermore, injection of cells at a high flow rate from the syringe into the marrow can result in turbulent flow, dissipation of energy and shear stress on the HPC cell membrane, which may result in cell disruption²¹. However, it is not known if cells having surface receptors for ligands found within the bone marrow endothelium²² and stroma²³ can be preferentially retained within the marrow using current IB techniques.

The porcine model was selected in this study because of its comparable relative marrow blood flow, cardiovascular physiology and anatomical size to humans²⁴. Using radiolabeled HPCs injected cells were tracked in vivo and conditions were optimized for retaining HPCs in the bone marrow using the IB route. Even though there is only partial species receptor-ligand homology with humans²⁵, human CD34+ cells were selected because they can traffic in pigs and the cell-labeling technique needed optimization for human application. Using ⁸⁹Zr cell-labeling, uptake in the bone marrow was compared with different IB methods utilizing a PET/CT scanner. IV injection of ⁸⁹Zr-Df alone revealed rapid renal excretion whereas with IV injection of ⁸⁹Zr labeled HPCs revealed extensive lung trapping. The injection needle was optimized to deliver the cells at right angles to the needle using side fenestrations. By measuring pressure, volume and flow rate while monitoring the location of injected cells, the optimal injection parameters were determined that resulted in maximum retention of HPCs in the bone marrow. It was demonstrated that high intra-marrow pressures, led to extravasation into surrounding muscle and rapid venous drainage, both undesirable for cell retention within the marrow. High intra-marrow pressures were also shown to elevate PASP possibly due to pulmonary embolism. It was also showed that when two insertions were made into the ipsilateral pelvic bone, the second injection often leaked through the first injection site regardless of pressure or distance between the sites. Thus, the optimal number of retained HPCs in the marrow is achieved when pressures are controlled by limiting the volume to 10 ml and the flow rate to <0.2 ml/min into a single site in the iliac bone

The successful labeling of the transferred HPCs with ⁸⁹Zr was pivotal for monitoring their fate following injection. This method utilized a very simple mixture of ⁸⁹Zr, protamine and the cells. As uptake of the ⁸⁹Zr-protamine complex by the cells was self-limited, only miniscule doses (approximately 10 μCi per 2×10⁸ hCD34+ cells) of radioactivity was taken up by the cells, enabling a high viability among labeled cells with little loss due to radiation effects. In fact, the viability of the labeled cells was comparable to the unlabeled cells. Our unpublished data of long-term (days) functional studies in other mononuclear cells labeled with ⁸⁹Zr suggests that cellular functions are unaffected by the labeling process. The ability to accurately detect and image with such a low dose of radioactivity greatly reduces the risk of time-dependent cytotoxicity seen with the larger doses of ¹¹¹In-oxine required for cell labeling²⁶. The sensitivity of PET/CT scanners enables this small dose to still be detected. ⁸⁹Zr has several advantages for this task. It has a sufficiently long half-life to envision tracking cells for up to a week. The physical properties of ⁸⁹Zr are such that it produces relatively high spatial resolution²⁷ compared to other positron emitters. Because PET cameras are highly sensitive and have relatively better resolution than SPECT cameras and because there is no background signal in the recipient, even very tiny amounts of radioactivity can be detected. Moreover, since PET images are superimposed on high resolution CT images, it is possible to localize the injected cells with a high degree of certainty.

There may be other advantages of utilizing this approach. In the largest human clinical trial of IB UCB HCT in acute leukemia, of 26 patients evaluable none developed grade III-IV acute GVHD. This is contrast to the 9% risk of severe acute GVHD reported in other UCB HCT clinical trials^{30,31}. This immunological advantage has been demonstrated in small mammal allogeneic transplant models. In a mismatched murine model of allogeneic HCT, C57BL/6 recipient mice received BALB/c donor bone marrow and lymphocytes either IB or IV. Bone marrow mesenchymal stromal cells (MSCs) were cultured from the femurs of mice in both groups. Polarization of Th-2 cells was strongly facilitated after co-culture with the MSCs from the IB recipients, while Th-1 cells were predominantly induced by co-culture with the MSCs from the IV recipients. Furthermore, a significant amount of TGF-β was detected in the culture supernatants of MSCs from the IB recipients of donor bone marrow and lymphocytes, but not in those from the IV recipients³². In a lapine model of haploidentical HCT, male offspring Japanese white rabbits received maternal donor bone marrow cells IB following whole body irradiation, either harvested by perfusion with a 1 ml IB aliquot or by aspiration with a corresponding large volume aliquot. The perfusion harvest bone marrow IB recipients developed a reduced incidence of GVHD, improved survival rate, and generated a persistent and stable chimera for at least 2 years³³. In another study, Brown Norway rats were the recipients of hind limbs from donor Fischer 344 rats together with an IB transplantation of donor bone marrow cells after fludarabine and low dose irradiation conditioning. There was complete donor lympho-hematopoietic engraftment in the recipient rats and the transplanted limbs were accepted for more than 1 year without any clinical signs of rejection in all recipients. Lymphocytes harvested from transplanted recipient rats showed tolerance to both donor-type and recipient-type major histocompatibility complex determinants in mixed lymphocyte reaction but maintained a preserved response against third party³⁴. Thus, successful IB HCT could have multiple advantages.

The work described herein describes a cell-labeling technique that can be used to optimize cell injection methods into the bone marrow, which should be readily translatable to clinical trials. Injection is also optimized by altering the needle design for IB cell transfers, so that cells are injected with sideways fenestrations. The needle also permits careful monitoring of pressure, volume and infusion rate. Using these two refinements, this IB technique could result in optimized results for engraftment and reducing the risk of GVHD. In this way, transferred HPCs may have a higher probability of successful engraftment than with conventional IV routes of administration.

Experiments as those described above will be repeated in non-human primates.

Methods and Materials

The experiments described herein were performed with, but not limited to, the following methods and materials.

Anesthesia, Monitoring and Porcine Pelvis IB Access

All animal procedures were approved by the institutional Animal Care and Use Committee (animal study proposal H-0233). Domestic swine (Sus scrofa domesticus) of both genders weighing between 38-70 kg were utilized. All experiments were conducted under general anesthesia; all animals were sedated with ketamine, midazolam and propofol prior to endotracheal intubation and general anesthesia was maintained with isoflurane. Analgesia was provided with bupivacaine injected locally and buprenorphine administered intravenously. Animals had continuous EKG, end-tidal CO2, oral temperature, non-invasive pulse oximetry, and intra-arterial blood pressure monitoring throughout each experiment. Intravenous vecuronium was used for paralysis during imaging. Internal jugular vein and internal carotid artery cannulation were performed after cervical cut-down. A Swan-Ganz catheter was inserted via the internal jugular vein, under fluoroscopic guidance, to measure pulmonary artery pressure. Foley bladder catheterization was performed via cystostomy in males and transurethrally in females.

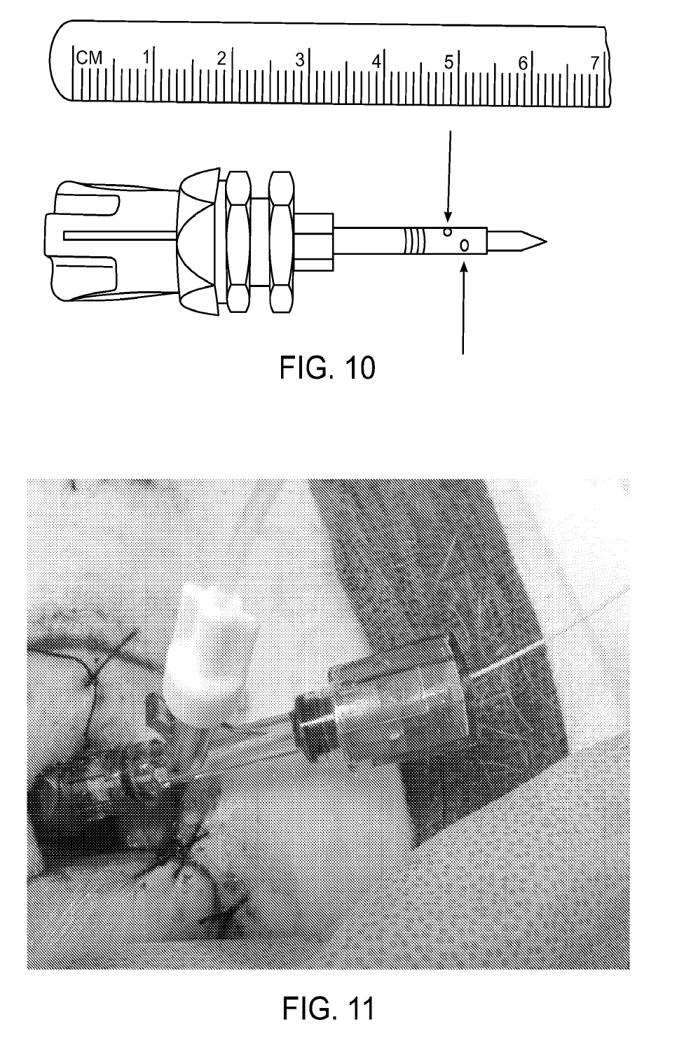

For the IB injection, the posterior superior iliac crest was exposed after minimal dissection. A truncated Jamshidi bone marrow needle (Cardinal Health, Dublin, Ohio) with a threaded base was used to establish IB access in animals and was also used for direct injection of iodinated contrast media. An ONCONTROL driver (Vidacare Corp., Shavano Park, Tex.) was used to insert modified shortened needles having distal fenestrations (FIG. 10) for IB HPC injections. A hemostasis screw type Y-connector (Cordis Corporation, Bridgewater, N.J.) was attached to the inserted bone marrow needle allowing passage of a 2Fr Millar MIKRO-TIP pressure catheter (ADInstruments, Colorado Springs, Colo.) into the bone marrow in order to measure injection pressure (FIG. 11). Intra-marrow cavity pressure was measured continuously during IB infusion and was compared to systemic arterial pressure, pulmonary arterial pressure, and electrocardiography on a PowerLab data acquisition system (ADInstruments, Colorado Springs, Colo.). Acquired hemodynamic data was analyzed using LABCHART 7 (ADInstruments, Colorado Springs, Colo.). At the conclusion of each experiment, animals were euthanized under anesthesia with intravenous potassium chloride.

Porcine Pelvic Vascular Imaging and Hemodynamic Monitoring

To evaluate flow through the bone marrow cavity, abdominal vessels were identified using iopamidol-370 contrast injected into the distal abdominal aorta via a 5Fr Glidecath (Terumo Medical Corporation, Somerset, N.J.) inserted in the internal carotid artery. To evaluate the anatomy of the veins, iopamidol-370 contrast was injected IB under anesthesia (boluses of 5 or 10 ml at rates of 0.1 to 2.5 ml/s) while scanning with 320-row detector CT (Aquilion ONE™, Toshiba Medical, Japan). Pulmonary artery, carotid artery and intra-marrow pressures were monitored and recorded continuously both during and after injection of contrast media. In select animals, vascular occluders (In Vivo Metric, Healdsburg, Calif.) were placed around the terminal branches of the abdominal aorta to selectively control flow through the internal or external iliac arteries, and around the iliac veins to control venous drainage from the pelvis. Selective catheterization of internal or external iliac arteries, with selective vessel occlusion, was performed to determine the dominant vessels supplying blood to marrow.

Cell Labeling

⁸⁹Zr was obtained from the local cyclotron facility utilizing the nuclear reaction Y(p, 2n) ⁸⁹Zr and an in-house GE PETtrace™ beam-line (GE Healthcare, Fairfield, Conn.) as described previously (Sato, et al). Produced 89Zr was provided as ⁸⁹Zr-oxalate with 1M oxalic acid in greater than 96% radiochemical yield. Prior to cell-labeling, ⁸⁹Zr-oxalate was neutralized with 2 M sodium bicarbonate and 2-[4-(2-hydroxyethyl)piperazin-1-yl]ethanesulfonic acid, then adjusted to 1 ml by water. ⁸⁹Zr cell-labeling was performed as previously described (Sato, et al). Briefly, the cells were incubated in the mixture of neutralized ⁸⁹Zr (1.85 to 3.7 MBq), protamine (40 μg/ml), and heparin (2 IU/ml) in phosphate-buffered saline (PBS) for 30 min at 37° C. The cells were washed with PBS twice and then with reconstituted deferoxamine (DF) mesylate USP (DESFERAL, Novartis Pharmaceuticals, East Hannover, N.J.) in PBS in order to remove the unbound ⁸⁹Zr. The amount of radioactivity associated with the cells before and after the labeling procedure was routinely determined with a WIZARD2 automatic γ-counter (Perkin Elmer, Waltham, Mass.)/dose calibrator (Capintec, Ramsey, N.J.). Cell viability was assessed immediately after the labeling procedure by trypan blue exclusion viability assay. The labeled cells were then immediately used. In some experiments, the number of surviving cells after the ⁸⁹Zr labeling was counted up to 14 h post-labeling.

In order to determine the retention of ⁸⁹Zr within the cells, one million CD34+ cells were cultured in RPMI medium and ⁸⁹Zr-activity associated with the cells and released to supernatant was counted by a γ-counter at various time points after the labeling.

Stem Cell IB Infusion and In Vivo PET/CT Imaging

To demonstrate the in vivo distribution of non-cell associated ⁸⁹Zr, ⁸⁹Zr-deferoxamine (⁸⁹Zr-Df) chelate (1 mCi in 10 ml solution) was injected IV into pigs followed immediately by PET/CT imaging. To determine the in vivo distribution of human CD34+ cells, all animals received 2×108 89Zr-labeled human CD34+(⁸⁹Zr-hCD34+) cells in 10 ml normal saline (8 to 10 μCi total radioactivity) injected either intravenously into the internal jugular vein, unilateral IB into the iliac crest, or intra arterially by selective catheterization of the internal iliac artery followed by PET/CT imaging. Animals were heparinzed to an ACT of 300 seconds or greater and were started with deferoxamine IV infusion (5 mg/kg/hr) to chelate any free ⁸⁹Zr that may potentially be released from fragmented cells.

PET scans were performed using a Gemini TF clinical PET/CT scanner (Philips Medical Systems, Andover, Mass.). Following injection, animals were imaged in 3-dimensional (3D) time-of-flight mode with a spatial resolution of 4.8 mm at the center of the field of view. The images were reconstructed using the default row-action maximum-likelihood algorithm iterative reconstruction, with standard corrections for random, scatter, attenuation, and normalization.

Image Analysis

Three volumes of interest (VOI) were manually drawn within the swine PET/CT scans. One VOI enclosing the intra-pelvic bone volume near the injection site, another VOI enclosing the extra-pelvic bone volume and the last VOI for the lung volume. The sum of measured activities of the three volumes equals the total injected radioactivity into the swine. Thus, the percent-injected dose (% ID) within each VOI was estimated by dividing each VOI by the sum of the three VOIs and multiplying by 100. The percent-injected dose for each of the three regions is reported herein.

### Example 2

### Maintaining Low IM Pressures is Critical to Maximizing Cellular Trapping in the Marrow Space Following Intra-Bone HPC Transplantation in Humans

Collection and Infusion

a) Human HPCs were mobilized from healthy volunteers using G-CSF. The HPCs were then positively selected for CD34+ cells using immuno-magnetic beads (Miltenyi Biotec, MA), and were cryopreserved.
b) Porcine bone marrow (BM) cells were aspirated (approximately 40 ml) from the iliac crest of swine. The BM cells were then were filtered and mononuclear cells (MNCs) were isolated using Ficoll-Paque™ with density gradient separation.

All animal procedures were conducted using domestic swine (Sus scrofa domesticus) on NHLBI Animal Use Committee approved protocols.

Intra-bone access in animals was initially achieved using the OnControl driver (Vidacare Corp. TX).

To evaluate flow through the marrow and venous drainage, direct intra-bone injection into the hemipelvis with iopamidol-370 contrast was performed under anesthesia with dynamic CT images acquired using a 320-detector row scanner (Aquilion One, Toshiba Medical, Japan). Human CD34+ and swine BM MNCs were labeled with Zirconium-89 (⁸⁹Zr), and were then assessed for viability, cell number, and the level of cellular radioactivity.

Radiolabeled cells were then injected into pigs either IV or directly through intra-bone infusion into the porcine pelvis at different infusion rates. Intramarrow (IM) pressures were measured continuously during intra-bone injection carried out using Millar catheters. IM pressures were acquired simultaneously with intra-arterial pressure and electrocardiography on a PowerLab data acquisition system (ADI Instruments, CO), and the data was analyzed using LabChart 7. After injection of labeled cells, positron emission tomography (PET) images were acquired for up to 180 minutes with a clinical PET/CT system (Gemini TF, Philips Medical Systems, MA) to assess cellular distribution and homing.

Peak IM pressures during bolus hand intra-bone injection were high, substantially exceeding systemic systolic arterial pressures. In contrast, IM pressures during slow intra-bone (less than 0.2 ml/min) infusion were significantly lower, remaining well below systemic blood pressure.

During manual sequential hand intra-bone injection of 5 ml aliquots of contrast at two different sites in the ipsilateral iliac crest, dynamic CT images revealed leakage from the initial access site after the first injection as well as immediate drainage into the ipsilateral iliac vein.

Following manual hand injection of ⁸⁹Zr labeled human CD34+ cells (⁸⁹Zr-hCD34+) given IV in swine via the external jugular vein, there was persistent PET activity noted in the lungs for up to 3 hrs.

Bolus hand intra-bone injection of ⁸⁹Zr labeled swine BM MNCs or ⁸⁹Zr-hCD34+ cells revealed PET activity in the iliac bone as well as activity in the lungs. Furthermore, PET activity following bolus hand intra-bone injection was also noted in surrounding tissues outside the bone when more than a single ipsilateral injection site was used.

In contrast, slow infusion of ⁸⁹Zr labeled swine BM MNCs or ⁸⁹Zr-hCD34+ cells resulted in PET activity that was limited to the iliac bone, indicating retention of cells within the marrow space with no leakage of cells to the lungs.

It was, thus, demonstrated that rapid hand infusion of HPCs into the pelvic bone results in cellular leakage out of the marrow space into the lungs. In contrast, slow intra-bone infusion of HPCs localizes cells to the bone marrow without leakage to the lungs. This data demonstrates that maintaining low IM pressures is critical to maximizing cellular trapping in the marrow space following intra-bone HPC transplantation in humans.

### INCORPORATION BY REFERENCE

All patents, publications, CAS numbers, and accession numbers mentioned in this specification are herein incorporated by reference to the same extent as if each independent patent and publication was specifically and individually indicated to be incorporated by reference.

### REFERENCES

1. Rahmim A, Zaidi H. PET versus SPECT: strengths, limitations and challenges. Nucl Med Commun. 2008; 29(3):193-207.
2. Lapidot T, Dar A, Kollet O. How do stem cells find their way home? Blood. 2005; 106(6):1901-1910.
3. Fischer U M, Harting M T, iimenez F, et al. Pulmonary Passage is a Major Obstacle for Intravenous Stem Cell Delivery: The Pulmonary First-Pass Effect. Stem Cells and Development. 2009; 18(5):683-691.
4. Wagner i E, Barker i N, DeFor T E, et al. Transplantation of unrelated donor umbilical cord blood in 102 patients with malignant and nonmalignant diseases: influence of CD34 cell dose and HLA disparity on treatment-related mortality and survival. Blood. 2002; 100(5):1611-1618.
5. Barker i N, Weisdorf D i, DeFor T E, et al. Transplantation of 2 partially HLA-matched umbilical cord blood units to enhance engraftment in adults with hematologic malignancy. Blood. 2005; 105(3):1343-1347.
6. Rocha V, Crotta A, Ruggeri A, et al. Double cord blood transplantation: extending the use of unrelated umbilical cord blood cells for patients with hematological diseases. Best Practice & Research Clinical Haematology. 2010; 23(2):223-229.
7. Xia Li, McDaniel i M, Yago T, Doeden A, McEver R P. Surface fucosylation of human cord blood cells augments binding to P-selectin and E-selectin and enhances engraftment in bone marrow. Blood. 2004; 104(10):3091-3096.
8. Frassoni F, Gualandi F, Podesta M, et al. Direct intrabone transplant of unrelated cord-blood cells in acute leukaemia: a phase I/II study. Lancet Oncology. 2008; 9(9):831-839.
9. Morrison M, Samwick A A. Intramedullary (sternal) transfusion of human bone marrow: Preliminary report. Journal of the American Medical Association. 1940; 115(20):1708-1711.
10. Hagglund H, Ringden O, Agren B, et al. Intraosseous compared to intravenous infusion of allogeneic bone marrow. Bone Marrow Transplantation. 1998; 21(4):331-335.
11. Brunstein C G, Barker i N, Weisdorf D i, et al. Intra-B M injection to enhance engraftment after myeloablative umbilical cord blood transplantation with two partially HLA-matched units. Bone Marrow Transplantation. 2009; 43(12):935-940.
12. Li Q, Hisha H, Yasumizu R, et al. Analyses of very early hemopoietic regeneration after bone marrow transplantation: Comparison of intravenous and intrabone marrow routes. Stem Cells. 2007; 25(5):1186-1194.
13. Mazurier F, Doedens M, Gan O I, Dick iE. Rapid myeloerythroid repopulation after intrafemoral transplantation of NOD-SCID mice reveals a new class of human stem cells. Nature Medicine. 2003; 9(7):959-963.
14. Omae M, Inaba M, Sakaguchi Y, et al. Long-term maintenance of donor-derived hematopoiesis by intra-bone marrow-bone marrow transplantation. Stem Cells and Development. 2008; 17(2):291-302.
15. Wang i F, Kimura T, Asada R, et al. SCID-repopulating cell activity of human cord blood-derived CD34(−) cells assured by intra-bone marrow injection. Blood. 2003; 101(8):2924-2931.
16. Kimura T, Matsuoka Y, Murakami M, et al. In vivo dynamics of human cord blood-derived CD34(−) SCID-repopulating cells using intra-bone marrow injection. Leukemia. 2010; 24(1):162-168.
17. Cameron i L, Fontanarosa P B, Passalaqua A M. A comparative study of peripheral to central circulation delivery times between intraosseous and intravenous injection using a radionuclide technique in normovolemic and hypovolemic canines. J Emerg Med. 1989; 7(2):123-127.
18. Schoffstall i M, Spivey W H, Davidheiser S, Lathers C M. Intraosseous crystalloid and blood infusion in a swine model. J Trauma. 1989; 29(3):384-387.
19. Hasan M Y, Kissoon N, Fiallos M, Abdelmoneim T, Johnson L, Murphy S. Incidence of fat and bone marrow embolism with the use of intraosseous infusion during cardio-pulmonary resuscitation. Pediatrics. 1997; 100(3):455-455.
20. Orlowski J P, Julius C J, Petras R E, Porembka D T, Gallagher J M. The Safety of Intraosseous Infusions—Risks of Fat and Bone-Marrow Emboli to the Lungs. Annals of Emergency Medicine. 1989; 18(10):1062-1068.
21. Zhang Z, Alrubeai M, Thomas C R. Estimation of Disruption of Animal-Cells by Turbulent Capillary Flow. Biotechnology and Bioengineering. 1993; 42(8):987-993.
22. Mazo I B, von Andrian U H. Adhesion and homing of blood-borne cells in bone marrow microvessels. J Leukoc Biol. 1999; 66(1):25-32.
23. Wagner W, Wein F, Roderburg C, et al. Adhesion of human hematopoietic progenitor cells to mesenchymal stromal cells involves CD44. Cells Tissues Organs. 2008; 188(1-2):160-169.
24. Upton R N. Organ weights and blood flows of sheep and pig for physiological pharmacokinetic modelling. J Pharmacol Toxicol Methods. 2008; 58(3):198-205.
25. Warrens A N, Simon A R, Theodore P R, Sykes M. Human-porcine receptor-ligand compatibility within the immune system: relevance for xenotransplantation. Xenotransplantation. 1999; 6(2):75-78.
26. Gholamrezanezhad A, Mirpour S, Bagheri M, Malekzadeh R, Saghari M. Cytotoxicity of 111In oxine on Mesenchymal Stem cells: A time dependent effect. Eur J Nucl Med Mol Imaging. 2008; 35:5317-5317.
27. Laforest R, Liu X. Image quality with non-standard nuclides in PET. Quarterly Journal of Nuclear Medicine and Molecular Imaging. 2008; 52(2):151-158.
28. Massollo M, Podesta M, Marini C, et al. Contact with the bone marrow microenvironment readdresses the fate of transplanted hematopoietic stem cells. Experimental Hematology. 2010; 38(10):968-977.
29. van Os R, Ausema A, Dontje B, van Riezen M, van Dam G, de Haan G. Engraftment of syngeneic bone marrow is not more efficient after intrafemoral transplantation than after traditional intravenous administration. Experimental Hematology. 2010; 38(11):1115-1123.
30. Thomson B G, Robertson K A, Gowan D, et al. Analysis of engraftment, graft-versus-host disease, and immune recovery following unrelated donor cord blood transplantation. Blood. 2000; 96(8):2703-2711.
31. Barker J N, Weisdorf D J, DeFor T E, Blazar B R, Miller J S, Wagner J E. Rapid and complete donor chimerism in adult recipients of unrelated donor umbilical cord blood transplantation after reduced-intensity conditioning. Blood. 2003; 102(5):1915-1919.
32. Miyake T, Inaba M, Fukui J, et al. Prevention of graft-versus-host disease by intrabone marrow injection of donor T cells: involvement of bone marrow stromal cells. Clin Exp Immunol. 2008; 152(1):153-162.
33. Cui Y, Nakamura S, Shi M, et al. A successful haploidentical bone marrow transplantation method in rabbits: perfusion method plus intra-bone marrow-bone marrow transplantation. Transpl Immunol. 2010; 24(1):33-39.
34. Esumi T, Inaba M, Ichioka N, Kushida T, Lida H, Ikehara S. Successful allogeneic leg transplantation in rats in conjunction with intra-bone marrow injection of donor bone marrow cells. Transplantation. 2003; 76(11): 1543-1548.

What is claimed is:

1. An intra-bone infusion system for performing a bone marrow transplant comprising:
an infusion source;
an intra-bone device having a proximal end for insertion into bone marrow, a distal end in connection with the infusion source, and a pressure sensor at the distal end for measuring intra-bone pressure; and
a mechanism in communication with the pressure sensor and the infusion source, the mechanism configured and arranged to continuously monitor intra-bone pressure measurements by the pressure sensor, and to automatically adjust infusion of agents from the infusion source and into the intra-bone device so as to maintain intra-bone pressure at levels not exceeding systemic blood pressure.

2. The intra-bone infusion system of claim 1, wherein
the intra-bone pressure does not exceed 25-30 mmHg.

3. The intra-bone infusion system of claim 1, wherein the intra-bone device comprises an access cannula and an infusion cannula, wherein the access cannula has a hub, an elongate portion extending from the hub, and a lumen extending through a length of the elongate portion, and the infusion cannula has a handle and an elongate portion insertable within the lumen of the access cannula.

4. The intra-bone infusion system of claim 3, wherein a distal end of the elongate portion of the access cannula is threaded.

5. The intra-bone infusion system of claim 3, wherein a distal end of the infusion cannula has one or more openings through which agents may be infused, and wherein the elongate portion of the infusion cannula is hollow.

6. The intra-bone infusion system of claim 3, wherein when the infusion cannula is inserted within the lumen of the access cannula, a distal end of the infusion cannula extends beyond a distal end of the access cannula.

7. The intra-bone infusion system of claim 6, wherein the pressure sensor is disposed along the outer surface of the infusion cannula.

8. The intra-bone infusion system of claim 3, wherein the intra-bone device further comprises a penetrator, the penetrator having a handle and an elongate portion extending from the handle, wherein the penetrator is insertable within the lumen of the access cannula.

9. The intra-bone infusion system of claim 1, wherein the pressure sensor is a fiberoptic probe.

10. The intra-bone infusion system of claim 1, wherein the intra-bone device is imagable by a number of imaging modalities including MRI, CT, fluoroscopy guidance imaging, optical guidance imaging, ultrasound guidance imaging, and the like.

11. The intra-bone infusion system of claim 1, further comprising a robotic guidance system, a CT guidance system, an MRI guidance system, a CT imaging system, a fluoroscopy guidance imaging system, an optical guidance imaging system, or an ultrasound guidance imaging system.

12. A method for infusing one or more agents into a target bone comprising:
inserting an intra-bone device into a target bone;
infusing one or more agents into the target bone while continuously measuring intra-bone pressure; and
adjusting a rate of infusion of the one or more agents to maintain intra-bone pressure at levels not exceeding systemic blood pressure.

13. (canceled)

14. The method of claim 12, wherein the one or more agents are selected from bone marrow, hematopoetic stem cells, mesenchymal stem cells, therapeutic agents and gene therapy vectors.

15. The method of claim 12, wherein the agent is a hematopoetic stem cell.

16. The method of claim 12, wherein the target bone is the pelvic bone.

17. The method of claim 12, carried out under MRI imaging, CT imaging, fluoroscopy guidance imaging, optical guidance imaging, ultrasound guidance imaging, and the like.

18. The method of claim 12, wherein one or more of the steps of inserting the intra-bone device, infusing one or more agents, and adjusting a rate of infusion are carried out robotically.

19. A method for increasing engraftment in a human subject undergoing a bone marrow transplant comprising:
inserting an intra-bone device into a target bone, wherein the intra-bone device is provided with a pressure sensor for measuring intra-bone pressure;
infusing one or more agents through the intra-bone device while continuously measuring intra-bone pressure; and
adjusting a rate of infusion of the one or more agents to maintain intra-bone pressure at levels not exceeding systemic blood pressure.

20. The method of claim 19, wherein at least about 85% engraftment is provided.

21. The method of claim 20, wherein the infusion volume of the one or more agents is between 5-15 ml.

22. The method of claim 20, wherein the rate of infusion is less than 0.2 ml/s.

23. The method of claim 20, further comprising the step of temporary occlusion of the iliac vein.
